(19) United States
(12) Patent Application Publication
(10) Pub. No.: US 2007/0244057 A1
(43) Pub. Date: Oct. 18, 2007

(54) Suppressing polyglutamine aggregation and toxicity

(76) Inventors: Henry Paulson, Iowa City, IA (US); Victor Miller, Jacksonville, FL (US)

Correspondence Address: VIKSNINS HARRIS & PADYS PLLP, P.O. BOX 111098, ST. PAUL, MN, 55111-1098, US

(21) Appl. No.: 11/656,048

(22) Filed: Jan. 22, 2007

Related U.S. Application Data
(60) Provisional application No. 60/760,435, filed on Jan. 20, 2006.

(51) Int. Cl. A61K 38/07; A61K 38/06; A61P 25/28; C12N 5/06; C12N 5/08; C12N 5/10

(52) U.S. Cl. 514/18; 435/375

(57) ABSTRACT

It has been discovered that CHIP suppresses polyglutamine aggregation and toxicity in transfected cell lines, primary neurons and a novel zebrafish model of disease. Accordingly, certain embodiments of the present invention provide methods for decreasing the formation of an inclusion or aggregation of a protein or for increasing the solubility of a protein in a cell, comprising increasing the amount of C-terminal heat shock protein 70-interacting protein (CHIP) or a functional subunit of CHIP in the cell. Additionally, certain embodiments of the present invention provide methods for treating a subject that has a neurodegenerative disease or preventing a neurodegenerative disease in a subject, comprising administering to the subject a treatment effective to increase the amount of CHIP, or a functional subunit thereof, in cells of the subject.

### RELATED APPLICATION

This patent document claims the benefit of priority of U.S. application Ser. No. 60/760,435, filed Jan. 20, 2006, which application is herein incorporated by reference.

### STATEMENT OF GOVERNMENT SUPPORT

Work related to this patent document was funded by the U.S. government (NIH Grants T32 GM08629, NS38712 and NS47535). The government may have certain rights in this patent document.

### BACKGROUND

The polyQ diseases are a group of neurodegenerative disorders caused by expansion of CAG trinucleotide repeats coding for polyQ. In the gene products, the polyQ region is the only feature shared by the polyQ disease proteins. They otherwise show no sequence homology, and they carry the glutamine stretch at different positions. The disorders develop when the length of the polyQ tract exceeds a threshold of 35-45 glutamines. PolyQ expansions induce protein aggregation and neuronal death. Evidence suggests that protein misfolding and aggregation play a crucial role in the pathogenesis.

Huntington's disease is an inherited neurodegenerative disease that is progressive and fatal. The disease-causing gene produces a protein that is toxic to certain brain cells. The protein contains abnormally long stretches of repeated glutamines and is prone to misfold and clump together and to form aggregates. The neuronal damage associated with Huntington's disease leads to movement disorders, psychiatric disturbances and cognitive decline. Thus, therapies useful for treating Huntington's disease and other neurodegenerative diseases, for example by decreasing protein aggregation and toxicity, are needed.

Human ataxin-3, the protein related to SCA3/MJD, is a ubiquitously expressed 41 kDa protein whose polyQ tract contains 12-40 glutamines in normal individuals and 55-84 glutamines in the pathogenic form. Ataxin-3 is found in the genomes of several species, from nematodes to humans, including plants.

### SUMMARY OF CERTAIN EMBODIMENTS OF THE INVENTION

It has been discovered that the C-terminal heat shock protein 70-interacting protein (CHIP) suppresses polyglutamine aggregation and toxicity. Accordingly, certain embodiments of the present invention provide methods for decreasing the formation of an inclusion in a cell, comprising increasing the amount of CHIP, or a function subunit thereof, in the cell. Certain embodiments of the present invention also provide methods for decreasing aggregation of a target protein in a cell, comprising increasing the amount of CHIP, or a functional subunit thereof, in the cell. In certain embodiments, the aggregation of the protein in the cell is decreased by 1%, 5%, 10%, 15%, 20%, 25%, 30%, 35%, 40%, 45%, 50%, 55%, 60%, 65%, 70%, 75%, 80%, 85%, 90%, 95%, or 100%. In certain embodiments, the target protein is a protein that comprises a polyglutamine repeat. In certain embodiments, the polyglutamine repeat has more than 46 glutamines. Certain embodiments of the present invention also provide methods for decreasing cell death, comprising increasing the amount of CHIP, or a functional subunit thereof, in a cell. In certain embodiments, the rate of death for a cell is decreased by 1%, 5%, 10%, 15%, 20%, 25%, 30%, 35%, 40%, 45%, 50%, 55%, 60%, 65%, 70%, 75%, 80%, 85%, 90%, 95%, or 100%. Certain embodiments of the present invention also provide methods for increasing the solubility of a target protein in a cell, comprising increasing the amount of CHIP, or a functional subunit thereof, in the cell. In certain embodiments, the solubility of the target protein in a cell is increased by 1%, 5%, 10%, 15%, 20%, 25%, 30%, 35%, 40%, 45%, 50%, 55%, 60%, 65%, 70%, 75%, 80%, 85%, 90%, 95%, or 100%. In certain embodiments, the amount of CHIP, or a functional subunit thereof, is increased by 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 500%, 800% or 1000%.

In certain embodiments of the invention, the functional subunit of CHIP comprises at least one (e.g., 1 or 2 or 3 or 4 or 5 or 6) tetratrico peptide repeat (TPR) domains. In certain embodiments of the invention, the function subunit of CHIP comprises two TPR domains. In certain embodiments of the invention, the function subunit of CHIP comprises three TPR domains. In certain embodiments of the invention, the function subunit of CHIP does not comprise an E4/U-box domain.

In certain embodiments of the invention, the target cell is a mammalian cell, such as a human cell. In certain embodiments of the invention, the target cell is a neuronal cell. The target cell may be in vitro or in vivo. In certain embodiments of the invention, the target cell is a neuron in a subject's brain.

In certain embodiments of the invention, the amount of CHIP is increased by introducing a vector comprising a nucleic acid encoding CHIP into the cell. In certain embodiments the vector is a viral vector (e.g., an adenoviral vector, an adeno-associated virus vector, a recombinant lentivirus or retrovirus vector) or a plasmid. In certain embodiments the amount of CHIP is increased by introducing CHIP protein into the cell.

Certain embodiments of the present invention provide methods for treating a subject that has a neurodegenerative disease, comprising administering to the subject a treatment effective to increase the amount of CHIP, or a functional subunit thereof, in cells of the subject. Certain embodiments of the present invention also provide methods for preventing a neurodegenerative disease in a subject, comprising administering to the subject a treatment effective to increase the amount of CHIP, or a functional subunit thereof, in cells of the subject. The amount of CHIP, or a functional subunit thereof, is increased by 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 500%, 800% or 1000%, as compared to the amount of CHIP present in the cell prior to treatment. In certain embodiments, the increase in the CHIP or the functional subunit of CHIP is effective to decrease formation of inclusions in cells of the subject, to decrease aggregation of a target protein in cells of the subject, to decrease cell death of cells of the subject, or to increase the solubility of a target protein in cells of the subject.

In certain embodiments of the invention, the subject is a mammal. In certain embodiments of the invention, the subject is not a human. In certain embodiments of the invention, the subject is a human. In certain embodiments of the invention, the subject is a male. In certain embodiments of the invention, the subject is a female. In certain embodiments of the invention, the neurodegenerative disease is a polyglutamine neurodegenerative disease. In certain embodiments of the invention, the neurodegenerative disease is Alzheimer's disease. In certain embodiments of the invention, the neurodegenerative disease is not Alzheimer's disease. In certain embodiments of the invention, the neurodegenerative disease is Huntington's disease.

In certain embodiments of the invention, the neurodegenerative disease is a prion disease, Alzheimer's Disease or related dementia, frontotemporal dementia with parkinsonism linked to chromosome 17 (FTDP-17), Parkinson's disease or related diseases, familial amyotrophic lateral sclerosis, spinocerebellar ataxia type 1, 2, 3, 6, 7, or 17, Huntington's Disease, spinal bulbar muscular atrophy, or dentatorubral-pallidoluyisian atrophy. Prion diseases are often called spongiform encephalopathies because of the post mortem appearance of the brain with large vacuoles in the cortex and cerebellum. Many mammalian species develop these diseases. Specific examples include: scrapie, TME (transmissible mink encephalopathy), CWD (chronic wasting disease), BSE (bovine spongiform encephalopathy), CJD (Creutzfeld-Jacob Disease), GSS (Gerstmann-Straussler-Scheinker syndrome), FFI (Fatal familial Insomnia), Kuru, or Alpers Syndrome.

In certain embodiments of the invention, the polyglutamine neurodegenerative disease is spinocerebellar ataxia types 1, 2, 3, 6, 7, or 17, Huntington's disease, spinal bulbar muscular atrophy, or dentatorubral-pallidoluyisian atrophy.

In certain embodiments of the invention, the cells are neuronal cells. In certain embodiments of the invention, the cells are neurons in the brain of a subject. In certain embodiments of the invention, the cells are in vitro. In certain embodiments of the invention, the cells are in vivo.

In certain embodiments of the invention, the target protein is a protein that comprises a polyglutamine repeat. In certain embodiments, the polyglutamine repeat is greater than 46 glutamines. In certain embodiments of the invention, the protein is a prion. In certain embodiments of the invention, the protein is a protein that is associated with (e.g., causes) a degenerative (e.g., a neurodegenerative) disease. In certain embodiments of the invention, the protein is polyQ, tau, pael-R, SOD1, ataxin-3, or firefly luciferase. In certain embodiments of the invention, the protein is not tau.

In certain embodiments of the invention, the increase in the CHIP, or the functional subunit of CHIP, is effective to decrease formation of inclusions in cells of the subject. In certain embodiments of the invention, the increase in the CHIP, or the functional subunit of CHIP, is effective to decrease aggregation of a target protein in cells of the subject. In certain embodiments of the invention, the increase in the CHIP, or the functional subunit of CHIP, is effective to decrease cell death of cells of the subject. In certain embodiments of the invention, the increase in the CHIP, or the functional subunit of CHIP, is effective to increase the solubility of a target protein in cells of the subject.

Certain embodiments of the present invention relate to increasing the amount of CHIP in cells (e.g., in vitro, in vivo, or ex vivo). CHIP can be increased using any method effective to increase CHIP. In certain embodiments of the invention, CHIP is increased by upregulating CHIP expression. In certain embodiments of the invention, CHIP is increased using gene therapy. In certain embodiments of the invention, CHIP is increased using gene transfer of plasmid DNA or by transduction with virus, e.g., a recombinant, expressing CHIP. In certain embodiments of the invention, the effects of CHIP are potentiated by using a treatment that affects (e.g., improves) the interaction of CHIP with a protein.

In certain embodiments of the invention, CHIP is increased using a pharmacological treatment. In certain embodiments of the invention, the treatment (e.g., a compound) is identified by screening a compound library such as the NINDS custom collection against the treatment. Further, a neural cell line or primary neurons could be arrayed in plates and exposed to the treatment and then CHIP levels could then be assessed by Western blot. Treatments that increased CHIP levels would then be identified. Further, following identification of the CHIP promoter, and a neural cell line that expresses a reporter gene (e.g., luciferase) under control of the CHIP promoter could be created. A screening strategy to identify treatments that upregulate CHIP expression could then be used to identify treatments that increase CHIP.

### BRIEF DESCRIPTION OF THE FIGURES

FIGS. 1A and 1B. CHIP suppresses aggregation of a mutant polyQ protein in cell culture. (A) Diagram of polyQ proteins used in this study and of CHIP showing TPR and E4/U-box domains. (B) Quantitation of visible inclusions in differentiated PC12 neural cells co-transfected with Q71-GFPu or Q56-GFP and the indicated plasmids. Graph depicts mean and SD of two independent experiments. Quantitation was performed 48 h after transfection. “Vector” indicates empty control plasmid, pcDNA3.

FIG. 2. Bar graph shows quantitation of SDS-resistant, GFP-Q82-Htt aggregates 3 minutes after detergent lysis. Percentage residual GFP fluorescence is shown relative to control cells co-transfected with GFP-Q82-Htt and control vector (set at 100%). Graph depicts means and SD of two independent experiments.

FIGS. 3A and 3B. CHIP rescues polyQ aggregation and toxicity in primary neurons. (A) Quantitation of inclusions in cortical neurons transfected with Q71-GFPu and the indicated CHIP variants. Cells were scored 48 h after transfection as having no inclusions or one or more inclusions. Bars depict mean and SD of four independent experiments. (B) Assessment of toxicity in cortical neurons transfected with Q71-GFPu and the indicated CHIP variants. Cells were scored at 48 and 72 h under brightfield and fluorescent illumination. Cells were identified as sick/dead if the cell body was rounded and displayed retracted or collapsed processes, or had an apoptotic, blebbed appearance. Bars depict mean and standard error of the mean (SEM) for two independent experiments performed in duplicate. Asterisks indicate significant difference between groups: **=P<0.01; *=P<0.02.

FIGS. 4A and 4B. Modeling features of polyQ disease in zebrafish. (A) Quantitation of dead embryos 24 h after fertilization following injection at the one cell stage with plasmids encoding the indicated polyQ-GFP fusions. Increasing polyQ length is associated with increased lethality. Bars depict mean and SD of five independent injections (>100 animals analyzed per condition). (B) Quantitation of inclusion formation in surviving embryos. Embryos were scored by a blinded observer as having 0, 1 to 10, 11 to 20, or more than 20 inclusions per embryo. Bars represent mean and SD of four independent injections (>50 animals were analyzed per condition).

FIGS. 5A and 5B. CHIP suppresses polyQ toxicity in developing zebrafish. Quantitation of embryo death 24 h after fertilization, following injection at the one cell stage of Q71-GFPu (5A) or GFP-Q82-Htt (5B) together with the indicated CHIP plasmids. Death is decreased by co-expression of WT-CHIP but not mutant CHIP-ΔTPR. Bars depict mean and SD of four independent injections for Q71-GFPu (>120 total animals analyzed) and three independent injections for GFP-Q82-Htt (>80 total animals analyzed). * indicates significant difference between groups (P<0.02).

FIGS. 6A and 6B. CHIP haplosufficiency accelerates the phenotype of HD transgenic mice. (A) Survival analysis for mice of the indicated genotypes (N=7 WT, 10 CHIP^{+/-}, 10 HD, 18 HD×CHIP^{+/-}). (B) Accelerating rotarod analysis for 14-15 week old female mice of the indicated genotypes (N=4 WT, 3 CHIP^{+/-}, 5 HD, 3 HD×CHIP^{+/-}).

FIGS. 7A, 7B and 7C. FIG. 7A compares survival of Q71-B mice. FIG. 7B gives the results of the rotarod testing, and FIG. 10C gives the results of open field testing.

### DETAILED DESCRIPTION

It has been discovered that the C-terminal Hsp70-interacting protein (CHIP) suppresses polyglutamine (polyQ) aggregation and toxicity in transfected cell lines, primary neurons and a novel zebrafish model of disease. CHIP's co-chaperone function was important for the suppression, indicating that CHIP acts to facilitate the solubility of mutant polyQ proteins through its interactions with chaperones. Conversely, Huntington disease transgenic mice that are haploinsufficient for CHIP displayed a markedly accelerated disease phenotype. Thus, based on these findings, CHIP appears to be an important mediator of the neuronal response to misfolded polyQ protein, and CHIP thus represents a therapeutic target for treating neurodegenerative diseases such as the polyQ neurodegenerative diseases, including Huntington disease.

The ability of CHIP to modulate the cellular response to mutant polyQ proteins using several complementary systems is herein described. In cell-based models, CHIP reduced accumulation of insoluble aggregates in a manner that involved the TPR domain, indicating that CHIP increases cellular capacity to maintain polyQ proteins in a soluble state. CHIP also reduced polyQ aggregation and toxicity in primary neurons in vitro. The ability of CHIP to modulate toxicity in zebrafish was also evaluated, taking advantage of several powerful features of this novel vertebrate system. Finally, inactivation of a single copy of endogenous CHIP markedly accelerated disease in a Huntington disease (HD) transgenic mouse model.

Thus, using complementary cellular and animal models, it is demonstrated herein that CHIP is an important component of the neuronal quality control (QC) machinery. In the various model systems described herein, CHIP levels determined the cellular ability to withstand the insults of polyQ aggregation and toxicity. Taken together, these results lead to a better understanding of the role of QC in polyQ disease pathogenesis and provide avenues for therapy for this incurable group of neurodegenerative disorders.

Neurodegenerative Diseases and CHIP

Huntington disease and other polyQ neurodegenerative diseases are characterized by neuronal accumulation of disease protein, indicating that the cellular ability to handle abnormal proteins may be compromised. As both a co-chaperone and ubiquitin ligase, CHIP links the two major arms of protein QC, molecular chaperones and the ubiquitin-proteasome system. The process of QC is crucial for neurons, which must function for decades in the face of high metabolic demands, environmental insults and age-related physiological changes. Genetic mutations can further predispose neurons to pathological failures of QC, manifesting as neurodegenerative disease. An important example is the group of dominantly inherited diseases caused by polyQ expansion, which include Huntington's disease (HD) and at least eight other neurodegenerative diseases (Taylor et al., (2002) Science 296:1991-1995).

PolyQ neurodegeneration is accompanied by the formation of neuronal inclusions that sequester molecular chaperones and proteasome components. Thus, perturbations in protein homeostasis may contribute to polyQ pathogenesis. Evidence suggests that the two central arms of QC, molecular chaperones and the ubiquitin-proteasome pathway (UPP), are taxed beyond physiological capacity in polyQ diseases. In light of this, components of the QC machinery have been manipulated in an effort to reduce polyQ toxicity. For example, overexpressing Hsp chaperones shows benefit in some model systems. However, in mouse models of disease, crosses to Hsp70-overexpressing mice have resulted in, at best, a modest therapeutic benefit (Cummings et al., (2001) Hum Mol Genet 10:1511-1518; Hansson et al., (2003) Brain Res 970:47-57; and Hay et al., (2004) Hum Mol Genet).

Molecular chaperones participate in nascent protein folding and the refolding of proteins damaged by physiological stress or mutations. If a native conformation cannot be achieved, futile refolding efforts by chaperones either continue or the protein is targeted for degradation. For many damaged or misfolded proteins, the principal route for this degradation is the UPP (Hartl F U, Hayer-Hartl M (2002) Science 295:1852-1858; Berke S J, Paulson H L (2003) Curr Opin Genet Dev 13:253-261). To triage proteins correctly between refolding and degradation, cells must possess molecular links between these two pathways (Cyr et al., (2002) Trends Biochem Sci 27:368-375).

CHIP is one such link. CHIP has three TPR domains that interact with the molecular chaperones Hsp70 and Hsp90, and an E4/U-box domain that interacts with the proteasome and confers E3 ubiquitin ligase activity on CHIP (D'Andrea et al., Trends Biochem Sci. 2003 December; 28(12):655-662; Das et al., EMBO J. 17 (1998), pp. 1192-1199; Zhang et al., Mol Cell. 2005 Nov. 23; 20(4):525-538; Ballinger et al., (1999) Mol Cell Biol 19:4535-4545; Jiang et al., (2001) J Biol Chem 276:42938-42944; Cyr et al., Trends Biochem Sci. 2002 July; 27(7):368-75). CHIP enhances refolding of some proteins while facilitating the ubiquitination and clearance of others (Connell et al., (2001) Nat Cell Biol 3:93-96; Meacham et al., (2001) Nat Cell Biol 3:100-105; Kampinga et al., (2003) Mol Cell Biol 23:4948-4958). CHIP can also regulate heat shock transcription factor 1 (HSF1), the principal transcription factor modulating chaperone expression during stress (Dai et al., (2003) Embo J 22:5446-5458). Thus, CHIP is strategically positioned within the cell's protein QC systems.

CHIP participates in triage decisions for specific substrate proteins (Connell et al., (2001) Nat Cell Biol 3:93-96). However, CHIP did not facilitate degradation of polyQ protein despite its known E3 ligase activity. It was originally described as a co-chaperone that converts Hsp70 activity from substrate folding to degradation. For example, for CFTR and several other substrates (Connell et al., (2001) Nat Cell Biol 3:93-96; Meacham et al., (2001) Nat Cell Biol 3:100-105), CHIP stimulates ubiquitination, either directly through its E3 ligase activity or in cooperation with other ubiquitin ligases. CHIP does not, however, enhance degradation of all substrates. For instance, CHIP has no effect on the degradation of apolipoprotein B48, luciferase or, in one study, the polyQ disease protein ataxin-3 (Meacham et al., (2001) Nat Cell Biol 3:100-105; Kampinga et al., (2003) Mol Cell Biol 23:4948-4958; Matsumoto et al., (2004) Embo J 23:659-669). In the case of luciferase, even inducing misfolding of the protein by heat denaturation does not enhance its degradation by CHIP. Instead, CHIP increases Hsp70-dependent refolding of luciferase by inhibiting the ATP hydrolysis cycle of Hsp70 (Kampinga et al., (2003) Mol Cell Biol 23:4948-4958; Ballinger et al., (1999) Mol Cell Biol 19:4535-4545). ATP-bound Hsp70 has an increased substrate on-rate which increases overall substrate loading. In the presence of increased CHIP, therefore, a greater fraction of cellular Hsp70 is bound to nascent polypeptide chains (Kampinga et al., (2003) Mol Cell Biol 23:4948-4958). CHIP, through this action, may enhance polyQ protein folding by increasing the probability that misfolded protein is bound to chaperones and eventually processed through multiple rounds of the chaperone cycle. Increased binding of Hsp70 to the surface of misfolded polyQ proteins would also be expected to retard oligomerization, which is postulated to be a key step in polyQ toxicity and aggregation (Sanchez et al., (2003) Nature 421:373-379). While the results presented herein with polyQ proteins are consistent with one of the known functions of CHIP, they suggest that CHIP's role in QC is quite complex, participating in differing aspects of protein triage depending on the specific substrate.

To extend the cell-based studies of CHIP presented herein, zebrafish were used as a fast and efficient vertebrate model system. These studies are the first to use zebrafish to model features of polyQ disease. Embryonic zebrafish replicate two features central to the human disease: polyQ length-dependent toxicity and aggregation. While these features are also readily recapitulated in other model systems, zebrafish embryos offer certain advantages for modeling polyQ disease: they develop rapidly and externally; can be produced quickly in large numbers; and are transparent, permitting direct analysis of organs, tissues and fluorescently tagged proteins. In addition, pathways and components of protein QC have been identified that are conserved from zebrafish to humans, including an HSF1-dependent stress response and a zebrafish ortholog of human CHIP (Wang et al., (2001) Genesis 30:195-197; Strausberg et al., (2002) Proc Natl Acad Sci USA 99:16899-16903). Antisense morpholinos can efficiently knock down expression of zebrafish genes raising the possibility of studying candidate suppressor and enhancer genes (Nasevicius et al., (2000) Nat Genet 26:216-220). Finally, a variety of tissue-specific promoters allow targeted transgene expression to select populations of brain cells. For example, using the neural specific GATA2 promoter (Meng et al., (1997) Proc Natl Acad Sci USA 94:6267-6272) to drive GFP-polyQ expression, it has been observed that aggregation in a subset of neurons in the developing zebrafish brain (unpublished results). These intrinsic advantages of zebrafish, coupled with the proof of principle demonstrated here, show that this is a simple, efficient system in which to model neurodegenerative disease mechanisms in a living vertebrate.

Growing experimental evidence implicates failures of protein QC in the pathogenesis of polyQ diseases. However, the cross of HD mice to CHIP haploinsufficient mice is only the second example in which impairment of a specific QC component has been shown to worsen the polyQ disease phenotype in mammals in vivo. The proteins most closely related to CHIP evolutionarily have not been identified as modifiers of polyQ aggregation in yeast or C. elegans screens, thus it striking that inactivation of one allele in mammals markedly accelerates the phenotype in an HD mouse model. This result likely reflects the particularly sensitive role that protein QC plays in mammalian neurons.

While the data presented herein collectively suggest that CHIP acts in concert with chaperones to directly mitigate polyQ toxicity, CHIP could also act indirectly. For example, CHIP regulates the degradation of p53, a protein recently implicated in HD pathogenesis (Bae et al., (2005) Neuron 47:29-41). Reduced CHIP levels in neurons could result in increased levels of p53 (or other substrates), thereby accelerating the disease phenotype. CHIP appears to participate at a crucial juncture in the neuron's handling of mutant polyQ proteins such that compromise of this process initiates a cascade of dysfunction severe enough to accelerate death of the animal. For this reason the HD×CHIP^{+/-} mouse provides a unique model in which to study the essential elements of protein QC that characterize the neuronal response to mutant polyQ proteins. CHIP and the proteins with which it associates can now be investigated as possible therapeutic targets in HD and other polyQ diseases.

Thus, while not a limitation of the present invention, the results presented herein suggest that CHIP acts primarily to enhance the neuronal capacity to maintain polyQ proteins in a soluble, nonaggregated state. CHIP appears to do so in a manner requiring an intact TPR domain, the region through which CHIP interacts with Hsp70 and Hsc70. Based on the studies presented herein, CHIP does not require an intact ubiquitin ligase domain. While these results do not rule out a role for the ubiquitin ligase activity of CHIP, they do suggest that chaperone interactions are likely to be important to its suppressor activity.

In summary, CHIP modulates aggregation and toxicity of polyQ disease proteins. A variety of complementary approaches to evaluate the effects of CHIP were used, beginning in immortalized cell culture followed by confirmatory studies in primary neurons, zebrafish and genetically modified mice. This tiered approach offers a systematic and rigorous method for evaluating future candidate modifiers. The fact that beneficial effects of increased CHIP activity were observed across several model systems strengthens its potential as a therapeutic target in neurodegenerative disease. The profound phenotypic effects caused by CHIP haploinsufficiency in an HD mouse model also raise interesting mechanistic questions about the role of CHIP and other multifunctional proteins in protein quality control.

Polypeptides and Proteins

“Polypeptides” and “proteins” are used interchangeably to refer to polymers of amino acids and do not refer to any specific lengths. These terms also include post-translationally modified proteins, for example glycosylated, acetylated, phosphorylated proteins and the like. Also included within the definition are, for example, proteins containing one or more analogs of an amino acid (including, for example, unnatural amino acids), proteins with substituted linkages, as well as other modifications known in the art, both naturally occurring and non-naturally occurring.

As used herein, the term “CHIP” includes variants of native CHIP. A “variant” of the protein is a protein that is substantially identical, but not necessarily completely identical to a native protein (e.g., is at least 50%, 55%, 60%, 65%, 70%, 75%, 80%, 85%, 90%, 91%, 92%, 93%, 94%, 95%, 96%, 97%, 98%, or 99% identical to native CHIP). A variant protein can be obtained by altering the amino acid sequence by insertion, deletion or substitution of one or more amino acid. The amino acid sequence of the protein is modified, for example by substitution, to create a polypeptide having substantially the same or improved qualities as compared to the native polypeptide. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cysteine, tyrosine, asparagine and glutamine having uncharged polar side chains; aspartate and glutamate having acidic side chains; and lysine, arginine, and histidine having basic side chains (Stryer, Biochemistry (2^{nd} ed.), W. H. Freeman and Co., San Francisco (1981) pp. 14-15; Lehninger, Biochemistry (2^{nd} ed.), Institute of Electrical & Electronics Enginee (1975), pp. 73-75).

It is known that variant polypeptides can be obtained based on substituting certain amino acids for other amino acids in the polypeptide structure in order to modify or improve biological activity. For example, through substitution of alternative amino acids, small conformational changes may be conferred upon a polypeptide that results in increased bioactivity. One can use the hydropathic index of amino acids in conferring interactive biological function on a polypeptide, wherein it is found that certain amino acids may be substituted for other amino acids having similar hydropathic indices and still retain a similar biological activity.

A variant of the invention may include amino acid residues not present in the corresponding native protein, or may include deletions relative to the corresponding native protein. A variant may also be a truncated fragment as compared to the corresponding native protein, i.e., only a portion of a full-length protein. Protein variants also include peptides having at least one D-amino acid.

The native CHIP protein has three tetratrico peptide repeat (TPR) domains and an E4/U-box domain. As used herein a “functional subunit of CHIP” includes a molecule consisting of one, two or three TRP domains, and may or may not contain an E4/U-box domain.

The CHIP or functional subunit of CHIP of the present invention may be expressed from isolated nucleic acid (DNA or RNA) sequences encoding the proteins. Amino acid changes from the native to the variant protein may be achieved by changing the codons of the corresponding nucleic acid sequence. Recombinant is defined as a peptide or nucleic acid produced by the processes of genetic engineering. It should be noted that it is well-known in the art that, due to the redundancy in the genetic code, individual nucleotides can be readily exchanged in a codon, and still result in an identical amino acid sequence.

It is known that such polypeptides can be obtained based on substituting certain amino acids for other amino acids in the polypeptide structure in order to modify or improve biological activity. For example, through substitution of alternative amino acids, small conformational changes may be conferred upon a polypeptide that results in increased biological activity. Alternatively, amino acid substitutions in certain polypeptide may be used to provide residues, which may then be linked to other molecules to provide peptide-molecule conjugates that retain sufficient properties of the starting polypeptide to be useful for other purposes.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated in intended for use in immunological embodiments. The greatest local average hydrophilicity of a “protein,” as governed by the hydrophilicity of its adjacent amino acids, correlates with its immunogenicity. U.S. Pat. No. 4,554,101. Accordingly, it is noted that substitutions can be made based on the hydrophilicity assigned to each amino acid.

In using either the hydrophilicity index or hydropathic index, which assigns values to each amino acid, it is preferred to conduct substitutions of amino acids where these values are ±2, with ±1 being particularly preferred, and those with in ±0.5 being the most preferred substitutions.

The variant protein has at least 50%, at least about 80% 81%, 82%, 83%, 84%, 85%, 86%, 87%, 88%, 89%, 90%, 91%, 92%, 93%, 94%, 95%, 96%, 96%, 97%, 98%, or even 99% (but less than 100%), contiguous amino acid sequence homology or identity to the amino acid sequence of a corresponding native protein.

The amino acid sequence of the variant polypeptide corresponds essentially to the native polypeptide's amino acid sequence. As used herein “correspond essentially to” refers to a polypeptide sequence that will elicit a biological response substantially the same as the response generated by the native protein. Such a response may be at least 60% of the level generated by the native protein, and may even be at least 80%, 87%, 90%, 95% or 99% of the level generated by native protein.

A variant of the invention may include amino acid residues not present in the corresponding native protein or deletions relative to the corresponding native protein. A variant may also be a truncated “fragment” as compared to the corresponding native protein, i.e., only a portion of a full-length protein. Protein variants also include peptides having at least one D-amino acid.

As used herein, a “transgenic,” “transformed,” or “recombinant” cell refers to a genetically modified or genetically altered cell, the genome of which includes a recombinant DNA molecule or sequence (“transgene”). For example, a “transgenic cell” can be a cell transformed with a “vector.” A “transgenic,” “transformed,” or “recombinant” cell thus refers to a host cell such as a bacterial or yeast cell into which a heterologous nucleic acid molecule has been introduced. The nucleic acid molecule can be stably integrated into the genome by methods generally known in the art. For example, “transformed,” “transformant,” and “transgenic” cells have been through the transformation process and contain a foreign or exogenous gene. The term “untransformed” refers to cells that have not been through the transformation process.

The term “transformation” refers to the transfer of a nucleic acid fragment into the genome of a host cell, or the transfer into a host cell of a nucleic acid fragment that is maintained extrachromosomally. A “transgene” refers to a gene that has been introduced into the genome by transformation. Transgenes may include, for example, genes that are heterologous or endogenous to the genes of a particular cell to be transformed. Additionally, transgenes may include native genes inserted into a non-native organism, or chimeric genes. The term “endogenous gene” refers to a native gene in its natural location in the genome of an organism. Such genes can be hyperactivated in some cases by the introduction of an exogenous strong promoter into operable association with the gene of interest. A “foreign” or an “exogenous” gene refers to a gene not normally found in the host cell but that is introduced by gene transfer.

The term “transfection” as used herein refers to the introduction of foreign DNA into eukaryotic cells. Transfection may be accomplished by a variety of means known to the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Vector; Transfection of Vectors

As used herein, the term “vector” is used in reference to nucleic acid molecules that can be used to transfer nucleic acid (e.g., DNA) segment(s) from one cell to another. The term “vehicle” is sometimes used interchangeably with “vector.” It is intended that any form of vehicle or vector be encompassed within this definition. For example, vectors include, but are not limited to viral particles, plasmids, and transposons.

“Vector” is defined to include, inter alia, any virus, plasmid, cosmid, phage or other construct in double or single stranded linear or circular form that may or may not be self transmissible or mobilizable, and that can transform prokaryotic or eukaryotic host either by integration into the cellular genome or exist extrachromosomally, e.g., autonomous replicating plasmid with an origin of replication. A vector can include a construct such as an expression cassette having a DNA sequence capable of directing expression of a particular nucleotide sequence in an appropriate host cell, comprising a promoter operably linked to the nucleotide sequence of interest that also is operably linked to termination signals.

An “expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense RNA, a nontranslated RNA in the sense or antisense direction, or a siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

Vectors may contain “viral replicons” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high copy number (up to 10⁴ copies/cell) in cells that express the appropriate viral T antigen. Vectors containing the replicons from bovine papillomavirus or Epstein-Barr virus replicate extrachromosomally at low copy number (about 100 copies/cell).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Additionally, multiple copies of the nucleic acid encoding enzymes may be linked together in the expression vector. Such multiple nucleic acids may be separated by linkers. The vector may be an adenoviral vector, an adeno-associated virus (AAV) vector, a retrovirus, or a lentivirus vector based on human immunodeficiency virus or feline immunodeficiency virus. The AAV and lentiviruses could confer lasting expression while the adenovirus would provide transient expression.

An expression cassette also typically includes sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

The term “wild type” refers to an untransformed cell, i.e., one where the genome has not been altered by the presence of the recombinant DNA molecule or sequence or by other means of mutagenesis. A “corresponding” untransformed cell is a typical control cell, i.e., one that has been subjected to transformation conditions, but has not been exposed to exogenous DNA.

“Isolated” DNA, RNA, peptides, polypeptides, or proteins are DNA, RNA, peptides polypeptides or proteins that are isolated or purified relative to other DNA, RNA, peptides, polypeptides, or proteins in the source material. For example, “isolated DNA” encoding the envelope protein (which would include cDNA) refers to DNA purified relative to DNA that encodes polypeptides other than the CHIP protein (or functional subunit of the CHIP protein).

Selectable and Screenable Markers

A vector may contain one or more selectable or screenable markers. Such markers are typically used to determine whether the vector has been successfully introduced into a host or target cell. A selectable marker is a gene whose expression substantially affects whether a cell will survive under particular controllable conditions. A selectable marker may provide for positive selection (cells with the marker are more likely to survive), negative selection (cells with the marker are less likely to survive), or both (the choice of environmental condition dictating whether positive or negative selection occurs).

Selectable markers include those that confer antibiotic resistance (or sensitivity), the ability to utilize a particular nutrient, and resistance (or sensitivity) to high (or low) temperature. Suitable selectable markers include the bacterial neomycin and hygromycin phosphotransferase resistance genes, which confers resistance to G418 and hygromycin, respectively, the bacterial gpt gene, which allows cells to grow in a medium containing mycophenolic acid, xanthine and aminopterin; the bacterial hisD gene that allows cells to grow in a medium lacking histidine but containing histidinol; the multidrug resistance gene mdr; the hprt and HSV thymidine kinase genes, which allow otherwise hprt- or tk-cells to grow in a medium containing hypoxanthine, amethopterin and thymidine, and the bacterial genes conferring resistance to puromycin or phleomycin. Positive or negative selection may require the use of a particular strain of host cell for the selection to be effective.

Screenable markers are genes that encode a product whose presence is readily detectable, directly or indirectly, but do not necessarily affect cell survival. The green fluorescent protein (GFP) is an example. Any cell surface protein not native to the host cell can be used as an immunoscreenable marker. Transformed cells may be segregated out by using a fluorescent antibody to the protein and a cell sorter. Many enzyme-encoding genes are useful as screenable markers, especially those encoding enzymes that can act upon a substrate to provide a colored or luminescent product. The luciferase and beta-galactosidase genes have been especially popular.

A dominant marker encodes an activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant activity. Examples of non-dominant selectable markers include the thymidine kinase (tk) gene that is used in conjunction with tk cell lines, the CAD gene that is used in conjunction with CAD-deficient cells and the mammalian hypoxanthine-guanine phosphoribosyl transferase (hprt) gene that is used in conjunction with hprt-cell lines.

Regulation of Gene Expression

The transgene(s) of the transgene vector, and the marker(s) and viral genes are expressed under the control of regulatory elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. A constitutive promoter is one that is always active at essentially a constant level.

Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types. For example, the SV40 early gene enhancer is very active in a wide variety of cell types from many mammalian species and has been widely used for the expression of proteins in mammalian cells. Two other examples of promoter/enhancer elements active in a broad range of mammalian cell types are those from the human elongation factor 1α gene and the long terminal repeats of the Rous sarcoma virus and the human cytomegalovirus.

As used herein, the term “promoter/enhancer” denotes a segment of DNA that contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of that gene is directed by the linked enhancer/promoter.

A regulatable promoter is one whose level of activity is subject to regulation by a regulatory molecule. An inducible promoter is one that is normally substantially inactive, but that is activated by the binding of an inducer to an operator site of the promoter. A repressible promoter is one that is normally active, but that is substantially inactivated by the binding of a repressor to an operator site of the promoter. Similar terminology applies to enhancers.

The inducer or repressor molecules are typically expressed only in particular tissues, at a particular developmental stage, or under particular environmental conditions (e.g., damage to the cell, infection, overproduction of a metabolite, absence of a nutrient). In the absence of an inducer an inducible promoter may be inactive or may produce a low level of activity. The level of activity in the presence of the inducer will be higher than the basal rate. A tightly inducible promoter is one whose basal level of activity is very low, e.g., less than 10% of its maximum inducible activity.

Different promoters may have different levels of basal activity in the same or different cell types. When two different promoters are compared in a given cell type in the absence of any inducing factors, if one promoter expresses at a higher level than the other it is said to have a higher basal activity.

The activity of a promoter and/or enhancer is measured by detecting directly or indirectly the level of transcription from the element(s). Direct detection involves quantitating the level of the RNA transcripts produced from that promoter and/or enhancer. Indirect detection involves quantitation of the level of a protein, often an enzyme, produced from RNA transcribed from the promoter and/or enhancer. A commonly employed assay for promoter or enhancer activity utilizes the chloramphenicol acetyltransferase (CAT) gene. A promoter and/or enhancer is inserted upstream from the coding region for the CAT gene on a plasmid; the plasmid is introduced into a cell line. The levels of CAT enzyme are measured. The level of enzymatic activity is proportional to the amount of CAT RNA transcribed by the cell line. This CAT assay therefore allows a comparison to be made of the relative strength of different promoters or enhancers in a given cell line. When a promoter is said to express at “high” or “low” levels in a cell line this refers to the level of activity relative to another promoter that is used as a reference or standard of promoter activity.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence that directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one that is one that is isolated from one gene and placed 3′ of another gene. A commonly used heterologous poly A signal is the SV40 poly A signal. The SV40 poly A signal is contained on a 237 bp Bam HI/Bcl I restriction fragment and directs both termination and polyadenylation.

The cytomegalovirus immediate early promoter-enhancer (CMV-IE) is a strong enhancer/promoter. Another strong promoter-enhancer for eukaryotic gene expression is the elongation factor 1α promoter enhancer.

The internal promoter for a transgene may be the promoter native to that transgene, or a promoter native to the target cell (or viruses infecting the target cell), or another promoter functional in the target cell.

The promoters and enhancers may be those exhibiting tissue or cell type specificity that can direct the transgene expression in the target cells at the right time(s). For example, a promoter to control human preproinsulin must be operable under control of carbohydrate in the liver. An example of such a promoter is the rat S-14 liver-specific promoter.

Promoters (and enhancers) may be naturally occurring sequences, or functional mutants thereof, including chimeras of natural sequences and mutants thereof. For example, a tissue-specific, development-specific, or otherwise regulatable element of one promoter may be introduced into another promoter.

Nucleic Acids

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “nucleotide sequence” refers to a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” or “polynucleotide” are used interchangeably and may also be used interchangeably with gene, cDNA, DNA and RNA encoded by a gene.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

“Naturally occurring” is used to describe a composition that can be found in nature as distinct from being artificially produced. For example, a nucleotide sequence present in an organism, which can be isolated from a source in nature and which has not been intentionally modified by a person in the laboratory, is naturally occurring.

“Functional RNA” refers to sense RNA, antisense RNA, ribozyme RNA, siRNA, or other RNA that may not be translated but yet has an effect on at least one cellular process.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters. Examples of promoters that may be used in the present invention include CMV, RSV, polII and polIII promoters.

A “5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

A “3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

A “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Constitutive expression” refers to expression using a constitutive promoter. “Conditional” and “regulated expression” refer to expression controlled by a regulated promoter.

“Operably-linked” refers to the association of nucleic acid sequences on a single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein.

The term “altered level of expression” refers to the level of expression in transgenic cells or organisms that differs from that of normal or untransformed cells or organisms.

“Antisense inhibition” refers to the production of antisense RNA transcripts capable of suppressing the expression of protein from an endogenous gene or a transgene.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity to the promoter sequences disclosed herein may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

Thus, the invention provides nucleic acid molecules and peptides that are substantially identical to the nucleic acid molecules and peptides described herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration is increased so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. For short nucleotide sequences (e.g., about 10 to 50 nucleotides), stringent conditions typically involve salt concentrations of less than about 1.5 M, less than about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

For the gene therapy methods, a person having ordinary skill in the art of molecular biology and gene therapy would be able to determine, without undue experimentation, the appropriate dosages and routes of administration of the polynucleotide, polypeptide, or expression vector used in the novel methods of the present invention.

The term “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) that stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

The selection and optimization of a particular expression vector for expressing a specific gene product in an isolated cell is accomplished by obtaining the gene, potentially with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

In one embodiment, vectors for gene therapy are viruses, such as replication-deficient viruses. Exemplary viral vectors are derived from: Harvey Sarcoma virus; ROUS Sarcoma virus, (MPSV); Moloney murine leukemia virus and DNA viruses (e.g., adenovirus).

The major advantage of using retroviruses, including lentiviruses, for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. While proliferation of the target cell is readily achieved in vitro, proliferation of many potential target cells in vivo is very low.

Yet another viral candidate useful as an expression vector for transformation of cells is the adenovirus, a double-stranded DNA virus. Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Finally, a third virus family adaptable for an expression vector for gene therapy are the recombinant adeno-associated viruses, specifically those based on AAV2, AAV4 and AAV5.

Thus, as will be apparent to one of ordinary skill in the art, a variety of suitable viral expression vectors are available for transferring exogenous genetic material into cells. The selection of an appropriate expression vector to express a therapeutic agent for a particular condition amenable to gene replacement therapy and the optimization of the conditions for insertion of the selected expression vector into the cell, are within the scope of one of ordinary skill in the art without the need for undue experimentation.

The present invention provides methods of decreasing the formation of an inclusion or aggregation of a protein or for increasing the solubility of a protein in a cell, comprising increasing the amount of C-terminal heat shock protein 70-interacting protein (CHIP) or a functional subunit of CHIP in the cell or in a mammal by administering a polynucleotide, polypeptide, expression vector, or cell. For the gene therapy methods, a person having ordinary skill in the art of molecular biology and gene therapy would be able to determine, without undue experimentation, the appropriate dosages and routes of administration of the polynucleotide, polypeptide, or expression vector used in the novel methods of the present invention.

The instant invention provides a cell expression system for expressing exogenous genetic material in a mammalian recipient. The expression system, also referred to as a “genetically modified cell,” comprises a cell and an expression vector for expressing the exogenous genetic material. The genetically modified cells are suitable for administration to a mammalian recipient, where they replace the endogenous cells of the recipient. Thus, the genetically modified cells may be non-immortalized and are non-tumorigenic.

According to one embodiment, the cells are transformed or otherwise genetically modified ex vivo. The cells are isolated from a mammal (for example, a human), transformed (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transformed or otherwise genetically modified in vivo. The cells from the mammalian recipient are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into anti-sense RNA, as well as a “heterologous gene” (i.e., a gene encoding a protein which is not expressed or is expressed at biologically insignificant levels in a naturally-occurring cell of the same type).

In the certain embodiments, the mammalian recipient has a condition that is amenable to gene augmentation or replacement therapy. As used herein, “gene augmentation or replacement therapy” refers to administration to the recipient of exogenous genetic material encoding a CHIP or a functional subunit of CHIP and subsequent expression of the administered genetic material in situ, thereby increasing the level of CHIP (or a functional subunit of CHIP) in the cell. Thus, the phrase “condition amenable to gene augmentation or replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid and/or protein components.

In an alternative embodiment, the expression vector is in the form of a plasmid, which is transferred into the target cells by one of a variety of methods: physical (e.g., microinjection, electroporation, scrape loading, microparticle bombardment or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

Retroviruses; Retroviral Vectors

The term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules that encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. There are several genera included within the family Retroviridae, including Cisternavirus A, Oncovirus A, Oncovirus B, Oncovirus C, Oncovirus D, Lentivirus, and Spumavirus. Some of the retroviruses are oncogenic (i.e., tumorigenic), while others are not. The oncoviruses induce sarcomas, leukemias, lymphomas, and mammary carcinomas in susceptible species. Retroviruses infect a wide variety of species, and may be transmitted both horizontally and vertically. They are integrated into the host DNA, and are capable of transmitting sequences of host DNA from cell to cell. This has led to the development of retroviruses as vectors for various purposes including gene therapy.

Retroviruses, including human foamy virus (HFV) and human immunodeficiency virus (HIV) have gained much recent attention, as their target cells are not limited to dividing cells and their restricted host cell tropism can be readily expanded via pseudotyping with vesicular stomatitis virus G (VSV-G) envelope glycoproteins.

Vector systems generally have a DNA vector containing a small portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle assembly.

The vector DNA is introduced into the packaging cell by any of a variety of techniques (e.g., calcium phosphate coprecipitation, lipofection, electroporation). The viral proteins produced by the packaging cell mediate the insertion of the vector sequences in the form of RNA into viral particles, which are shed into the culture supernatant.

A major limitation in the use of many commonly used retroviral vectors in gene transfer is that many of the vectors are restricted to dividing cells. If a non-dividing cell is the target cell, then a lentivirus, which is capable of infecting non-dividing cells are provided. Alternatively, for cells that are naturally dividing or stimulated to divide by growth factors, murine leukemia virus (MLV) vectors are suitable delivery systems.

In addition to simple retroviruses like MLV, lentiviruses can also be used as the vector. As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, that causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

Lentiviruses including HIV, SIV, FIV and equine infectious anemia virus (EIAV) depend on several viral regulatory genes in addition to the simple structural gag-pol-env genes for efficient intracellular replication. Thus, lentiviruses use more complex strategies than classical retroviruses for gene regulation and viral replication, with the packaging signals apparently spreading across the entire viral genome. These additional genes display a web of regulatory functions during the lentiviral life cycle. For example, upon HIV-1 infection, transcription is upregulated by the expression of Tat through interaction with an RNA target (TAR) in the LTR. Expression of the full-length and spliced mRNAs is then regulated by the function of Rev, which interacts with RNA elements present in the gag region and in the env region (RRE). Nuclear export of gag-pol and env mRNAs is dependent on the Rev function. In addition to these two essential regulatory genes, a list of accessory genes, including vif, vpr, vpx, vpu, and nef, are also present in the viral genome and their effects on efficient virus production and infectivity have been demonstrated, although they are not absolutely required for virus replication.

A “source” or “original” retrovirus is a wild-type retrovirus from which a pseudotyped retrovirus is derived, or is used as a starting point, during construction of the packaging or transgene vector, for the preparation of one or more of the genetic elements of the vector. The genetic element may be employed unchanged, or it may be mutated (but not beyond the point where it lacks a statistically significant sequence similarity to the original element). A vector may have more than one source retrovirus, and the different source retroviruses may be, e.g., MLV, FIV, HIV-1 and HIV-2, or HIV and SIV. The term “genetic element” includes but is not limited to a gene.

A cognate retrovirus is the wild-type retrovirus with which the vector in question has the greatest percentage sequence identity at the nucleic acid level. Normally, this will be the same as the source retrovirus. However, if a source retrovirus is extensively mutated, it is conceivable that the vector will then more closely resemble some other retrovirus. It is not necessary that the cognate retrovirus be the physical starting point for the construction; one may choose to synthesize a genetic element, especially a mutant element, directly, rather than to first obtain the original element and then modify it. The term “cognate” may similarly be applied to a protein, gene, or genetic element (e.g., splice donor site or packaging signal). When referring to a cognate protein, percentage sequence identities are determined at the amino acid level.

The term “cognate” retrovirus may be difficult to interpret in the extreme case, i.e., if all retroviral genetic elements have been replaced with surrogate non-lentiviral genetic elements. In this case, the source retrovirus strain mentioned previously is arbitrarily considered to be the cognate retrovirus.

The term “replication” as used herein in reference to a virus or vector, refers not to the normal replication of proviral DNA in a chromosome as a consequence of cell reproduction, or the autonomous replication of a plasmid DNA as a result of the presence of a functional origin of replication. Instead “replication” refers to the completion of a complete viral life cycle, wherein infectious viral particles containing viral RNA enter a cell, the RNA is reverse transcribed into DNA, the DNA integrates into the host chromosome as a provirus, the infected cell produces virion proteins and assembles them with full length viral genomic RNA into new, equally infectious particles.

The term “replication-competent” refers to a wild-type virus or mutant virus that is capable of replication, such that replication of the virus in an infected cell result in the production of infectious virions that, after infecting another, previously uninfected cell, causes the latter cell to likewise produce such infectious virions. The present invention contemplates the use of replication-defective virus.

As used herein, the term “attenuated virus” refers to any virus (e.g., an attenuated lentivirus) that has been modified so that its pathogenicity in the intended subject is substantially reduced. The virus may be attenuated to the point it is nonpathogenic from a clinical standpoint, i.e., that subjects exposed to the virus do not exhibit a statistically significant increased level of pathology relative to control subjects.

The present invention contemplates the preparation and use of a modified retrovirus. In some embodiments, the retrovirus is an mutant of murine leukemia virus, human immunodeficiency virus type 1, human immunodeficiency virus type 2, feline immunodeficiency virus, simian immunodeficiency virus, visna-maedi, caprine arthritis-encephalitis virus, equine infectious anemia virus, and bovine immune deficiency virus, or a virus comprised of portions of more than one retroviral species (e.g., a hybrid, comprised of portions of MLV, FIV, HIV-1 and HIV-2, or HIV-1 and/or SIV).

A reference virus is a virus whose genome is used in describing the components of a mutant virus. For example, a particular genetic element of the mutant virus may be said to differ from the cognate element of the reference virus by various substitutions, deletions or insertions. It is not necessary that the mutant virus actually be derived from the reference virus.

A reference FIV sequence is found in Talbott et al., Proc. Natl. Acad. Sci. USA, 86:5743-7 (1989). In certain embodiments, a three-plasmid transient transfection method can be used to produce replication incompetent pseudotyped retroviruses (e.g., FIV).

Retroviral Vector System

The present invention contemplates a retroviral gene amplification and transfer system comprising a transgene vector, one or more compatible packaging vectors, an envelope vector, and a suitable host cell. The vectors used may be derived from a retrovirus (e.g., a lentivirus). Retrovirus vectors allow (1) transfection of the packaging vectors and envelope vectors into the host cell to form a packaging cell line that produces essentially packaging-vector-RNA-free viral particles, (2) transfection of the transgene vector into the packaging cell line, (3) the packaging of the transgene vector RNA by the packaging cell line into infectious viral particles, and (4) the administration of the particles to target cells so that such cells are transduced and subsequently express a transgene.

Either the particles are administered directly to the subject, in vivo, or the subject's cells are removed, infected in vitro with the particles, and returned to the body of the subject.

The packaging vectors and transgene vectors of the present invention will generate replication-incompetent viruses. The vectors chosen for incorporation into a given vector system of the present invention are such that it is not possible, without further mutation of the packaging vector(s) or transgene vector, for the cotransfected cells to generate a replication-competent virus by homologous recombination of the packaging vector(s) and transgene vector alone. The envelope protein used in the present system can be a retroviral envelope, a synthetic or chimeric envelope, or the envelope from a non-retroviral enveloped virus (e.g., baculovirus).

As used herein, the term “packaging signal” or “packaging sequence” refers to sequences located within the retroviral genome or a vector that are required for, or at least facilitate, insertion of the viral or vector RNA into the viral capsid or particle. The packaging signals in an RNA identify that RNA as one that is to be packaged into a virion. The term “packaging signal” is also used for convenience to refer to a vector DNA sequence that is transcribed into a functional packaging signal. Certain packaging signals may be part of a gene, but are recognized in the form of RNA, rather than as a peptide moiety of the encoded protein.

The key distinction between a packaging vector and a transgene vector is that in the packaging vector, the major packaging signal is inactivated, and, in the transgene vector, the major packaging signal is functional. Ideally, in the packaging vector, all packaging signals would be inactivated, and, in the transgene vector, all packaging signals would be functional. However, countervailing considerations, such as maximizing viral titer, or inhibiting homologous recombination, may lend such constructs less desirable.

A packaging system is a vector, or a plurality of vectors, which collectively provide in expressible form all of the genetic information required to produce a virion that can encapsidate suitable RNA, transport it from the virion-producing cell, transmit it to a target cell, and, in the target cell, cause the RNA to be reverse transcribed and integrated into the host genome in a such a manner that a transgene incorporated into the aforementioned RNA can be expressed. However, the packaging system must be substantially incapable of packaging itself. Rather, it packages a separate transgene vector.

In the present invention, the packaging vector will provide functional equivalents of the gag and pol genes (a “GP” vector). The env gene(s) will be provided by the envelope vector. In theory, a three vector system (“G”, “P”, and “E” vectors) is possible if one is willing to construct distinct gag and pol genes on separate vectors, and operably link them to different regulatable promoters (or one to a regulatable and the other to a constitutive promoter) such that their relative levels of expression can be adjusted appropriately.

A packaging cell line is a suitable host cell transfected by a packaging system that, under achievable conditions, produces viral particles. As used herein, the term “packaging cell lines” is typically used in reference to cell lines that express viral structural proteins (e.g., gag, pol and env), but do not contain a packaging signal. For example, a cell line has been genetically engineered to carry at one chromosomal site within its genome, a 5′-LTR-gag-pol-3′-LTR fragment that lacks a functional psi⁺ sequence (designated as Δ-psi), and a 5′-LTR-env-3′-LTR fragment that is also Δ-psi located at another chromosomal site. While both of these segments are transcribed constitutively, because the psi⁺ region is missing and the viral RNA molecules produced are less than full-size, empty viral particles are formed.

If a host cell is transfected by the packaging vector(s) alone, it produces substantially only viral particles without the full-length packaging vector. In one example, less than 10% of the viral particles produced by the packaging cell contain full length packaging vector-derived RNA. However, since the packaging vector lacks a functional primer binding site, even if these particles infect a new cell, the packaging vector RNA will not be reverse transcribed back into DNA and therefore the new cell will not produce virion. Thus, by itself, the packaging vector is a replication-incompetent virus.

In some embodiments, the packaging cell and/or cell line contains a transgene vector. The packaging cell line will package the transgene vector into infectious particles. Such a cell line is referred to herein as a “transgenic virion production cell line.”

It is contemplated that packaging may be inducible, as well as non-inducible. In inducible packaging cells and packaging cell lines, retroviral particles are produced in response to at least one inducer. In non-inducible packaging cell lines and packaging cells, no inducer is required in order for retroviral particle production to occur.

The packaging vectors necessarily differ from wild-type, replication-competent retroviral genomes by virtue of the inactivation of at least one packaging signal of the cognate wild-type genome. More than one packaging signal may be inactivated. In one example, only the retroviral genes provided by the packaging vector are those encoding structural, or essential regulatory, proteins.

Methods of Preparing Retroviral Vectors

Recombinant retroviruses can be produced by a number of methods. One method is the use of packaging cell lines. The packaging cells are provided with viral protein-coding sequences, such as encoded on two three. The plasmids encode all proteins necessary for the production of viable retroviral particles and encode a RNA viral construct that carries the desired gene (e.g., CHIP), along with a packaging signal (Psi) that directs packaging of the RNA into the retroviral particles.

Alternatively, the mutated retroviral genome can be transfected into cells using commonly known transfection methods such as calcium chloride or electroporation.

The retroviral vector may also include an appropriate selectable marker. Examples of selectable markers that may be utilized in either eukaryotic or prokaryotic cells, include but are not limited to, the neomycin resistance marker (neo), the ampicillin resistance marker (amp), the hygromycin resistance marker (hygro), the multidrug resistance (mdr) gene, the dihydrofolate reductase (dhfr) gene, the beta-galactosidase gene (lacZ), and the chloramphenicol acetyl transferase (CAT) gene.

Cells transfected with cDNAs encoding a retrovirus genome or infected with retrovirus particles can be cultured to produce virions or virus particles. Virus particle-containing supernatant can be collected. The virus particles can be concentrated by centrifuging the supernatant, pelleting the virus and by size exclusion chromatography. Pharmaceutical compositions containing virus particles can also be resuspended in pharmaceutically acceptable liquids or carriers such as saline.

Retroviral Gene Transfer

The retrovirus particles described above can infect cells by the normal infection pathway as along as recognition of the target cell receptor, fusion and penetration into the cell all occur. All eukaryotic cells are contemplated for infection by the recombinant virions. For example, the cells used in the present invention can include cells from vertebrates (e.g., human cells).

The vectors of the present invention can be used in vivo with a number of different tissue types. Examples include airway epithelia, liver, and neural (e.g., central nervous system) cells. Methods of preparing and administering retroviral particles in gene therapy commonly known to the skilled artisan may be used.

Therapeutic genes or nucleic acid molecules are under the control of a suitable promoter. Suitable promoters, which may be employed, include, but are not limited to adenoviral promoters, the cytomegalovirus promoter, the Rous sarcoma virus (RSV) promoter, the respiratory syncytial virus promoter, inducible promoters such as the metallothionein promoter, heat shock promoters, or the gene's own natural promoter. It is to be understood however, that the scope of the present invention is not to be limited to specific foreign genes or promoters.

Most gene therapy is administered to cells ex vivo. The cells receiving such gene therapy treatment may be exposed to the retrovirus particles in combination with a pharmaceutically acceptable carrier suitable for administration to a patient. The carrier may be a liquid carrier (for example, a saline solution), or a solid carrier such as an implant or microcarrier beads. In employing a liquid carrier, the cells may be introduced intravenously, subcutaneously, intramuscularly, intraperitoneally, intralesionally, etc. In yet another embodiment, the cells may be administered by transplanting or grafting the cells. Lipid destabilizers, such as thiocationic lipids, can be utilized in admixture with the viral vector or liposomal vector to increase infectivity.

Although most current gene therapy protocols involve ex vivo transfection of cells, the vectors disclosed would permit in vivo treatment of a subject, such as a human patient, as well as ex vivo utilization. For example, ex vivo therapy requires that cells such as hepatocytes be removed from the patient, transduced with the retroviral particle containing the desired nucleic acid molecule, and then transplanted back into the patient. In vivo therapy would allow direct infusion of the gene therapy vector, without the intervening steps and the complications that they raise. Moreover, this will allow access to tissues that may not have been good candidates for ex vivo gene therapy.

Host Cells, Target Cells and Organisms

The host cell is a cell into which a vector of interest may be introduced and wherein it may be replicated, and, in the case of an expression vector, in which one or more vector-based genes may be expressed.

The transgene vector may be administered to a target organism by any route that will permit it to teach the target cells. Such route may be, e.g., intravenous, intratracheal, intracerebral, intramuscular, subcutaneous, or, with an enteric coating, oral. Alternatively, target cells may be removed from the organism, infected, and they (or their progeny) returned to the organism. Or the transgene vector may simply be administered to target cells in culture.

According to one aspect of the invention, a cell expression system for expressing a therapeutic agent in a mammalian recipient is provided. The expression system (also referred to herein as a “genetically modified cell”) comprises a cell and an expression vector for expressing the therapeutic agent. Expression vectors of the instant invention include, but are not limited to, viruses, plasmids, and other vehicles for delivering heterologous genetic material to cells. Accordingly, the term “expression vector” as used herein refers to a vehicle for delivering heterologous genetic material (i.e., a nucleic acid encoding CHIP or a functional subunit of CHIP) to a cell. In particular, the expression vector is a recombinant adenoviral, adeno-associated virus, or lentivirus or retrovirus vector.

The expression vector further includes a promoter for controlling transcription of the heterologous gene. The promoter may be an inducible promoter. The expression system is suitable for administration to the mammalian recipient. The expression system may comprise a plurality of non-immortalized genetically modified cells, each cell containing at least one recombinant gene encoding at least one therapeutic agent.

The cell expression system can be formed ex vivo or in vivo. To form the expression system ex vivo, one or more isolated cells are transduced with a virus or transfected with a nucleic acid or plasmid in vitro. The transduced or transfected cells are thereafter expanded in culture and thereafter administered to the mammalian recipient for delivery of the therapeutic agent in situ. The genetically modified cell may be an autologous cell. i.e. the cell is isolated from the mammalian recipient. The genetically modified cell(s) are administered to the recipient by, for example, implanting the cell(s) or a graft (or capsule) including a plurality of the cells into a cell-compatible site of the recipient.

According to yet another aspect of the invention, a method for treating a mammalian recipient in vivo is provided. The method includes introducing an expression vector for expressing a heterologous gene product into a cell of the patient in situ. To form the expression system in vivo, an expression vector for expressing the therapeutic agent is introduced in vivo into target location of the mammalian recipient by, for example, intraperitoneal injection or injection directly into the brain.

According to yet another aspect of the invention, a method for treating a mammalian recipient in vivo is provided. The method includes introducing the recombinant CHIP protein into the tissues of the patient in vivo. The therapeutic agent is introduced in vivo into target location of the mammalian recipient by, for example, a pump to provide continuous delivery into brain ventricles.

The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

According to yet another embodiment, a pharmaceutical composition is disclosed. The pharmaceutical composition comprises a plurality of the above-described genetically modified cells or polypeptides and a pharmaceutically acceptable carrier. The pharmaceutical composition may be for treating a condition amenable to gene replacement therapy and the exogenous genetic material comprises a heterologous gene encoding a therapeutic agent for treating the condition. The pharmaceutical composition may contain an amount of genetically modified cells or polypeptides sufficient to deliver a therapeutically effective dose of the therapeutic agent to the patient. Exemplary conditions amenable to gene replacement therapy are described below.

According to another aspect of the invention, a method for forming the above-described pharmaceutical composition is provided. The method includes introducing an expression vector for expressing a heterologous gene product into a cell to form a genetically modified cell and placing the genetically modified cell in a pharmaceutically acceptable carrier.

According to still another aspect of the invention, a cell graft is disclosed. The graft comprises a plurality of genetically modified cells attached to a support, which is suitable for implantation into the mammalian recipient. The support may be formed of a natural or synthetic material.

According to still another aspect of the invention, an encapsulated cell expression system is disclosed. The encapsulated expression system comprises a plurality of genetically modified cells contained within a capsule, which is suitable for implantation into the mammalian recipient. The capsule may be formed of a natural or synthetic material. The capsule containing the plurality of genetically modified cells may be implanted in the peritoneal cavity, the brain or ventricles in the brain, or into the specific disease site.

According to still another aspect of the invention, a protein delivery method is disclosed. The protein is purified from genetically modified cells and then placed into the mammalian recipient. The purified protein is placed into the brain, into the peritoneum, or into the specific disease site.

Methods for Introducing Genetic Material into Cells

The exogenous genetic material (e.g., a cDNA encoding one or more therapeutic proteins) is introduced into the cell ex vivo or in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-facilitated microparticle bombardment. Strontium phosphate DNA co-precipitation is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs), which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, transgene and/or signal sequence is deemed to be within the scope of one of ordinary skill in the art without undue experimentation.

The therapeutic agent can be targeted for delivery to an extracellular, intracellular or membrane location. If it is desirable for the gene product to be secreted from the cells, the expression vector is desired to include and appropriate secretion “signal” sequence for secreting the therapeutic gene product from the cell to the extracellular milieu. If it is desirable for the gene product to be retained within the cell, this secretion signal sequence is omitted. In a similar manner, the expression vector can be constructed to include “retention” signal sequences for anchoring the therapeutic agent within the cell plasma membrane. For example, all membrane proteins have hydrophobic transmembrane regions, which stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

The selection and optimization of a particular expression vector for expressing a specific gene product in an isolated cell is accomplished by obtaining the gene, potentially with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

In one embodiment, vectors for cell gene therapy are viruses, such as replication-deficient viruses. Exemplary viral vectors are derived from Harvey Sarcoma virus, ROUS Sarcoma virus, (MPSV), Moloney murine leukemia virus and DNA viruses (e.g., adenovirus).

Replication-deficient retroviruses, including the recombinant lentivirus vectors, are neither capable of directing synthesis of virion proteins or making infectious particles. Accordingly, these genetically altered retroviral expression vectors have general utility for high-efficiency transduction of genes in cultured cells, and specific utility for use in the method of the present invention. The lentiviruses, with their ability to transduce nondividing cells, have general utility for transduction of hepatocytes, cells in cerebrum, cerebellum and spinal cord, and also muscle and other slowly or non-dividing cells. Such retroviruses further have utility for the efficient transduction of genes in vivo. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in Kriegler, M. Gene Transfer and Expression, A Laboratory Manual, W.H. Freeman Co, New York, (1990) and Murray, E. J., ed. Methods in Molecular Biology, Vol. 7, Humana Press Inc., Clifton, N.J., (1991).

The major advantage of using retroviruses, including lentiviruses, for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. While proliferation of the target cell is readily achieved in vitro, proliferation of many potential target cells in vivo is very low.

Yet another viral candidate useful as an expression vector for transformation of cells is the adenovirus, a double-stranded DNA virus. The adenovirus is frequently responsible for respiratory tract infections in humans.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Finally, a third virus family adaptable for an expression vector for gene therapy are the recombinant adeno-associated viruses, specifically those based on AAV2, AAV4 and AAV5.

Thus, as will be apparent to one of ordinary skill in the art, a variety of suitable viral expression vectors are available for transferring exogenous genetic material into cells. The selection of an appropriate expression vector to express a therapeutic agent for a particular condition amenable to gene replacement therapy and the optimization of the conditions for insertion of the selected expression vector into the cell, are within the scope of one of ordinary skill in the art without the need for undue experimentation.

In an alternative embodiment, the expression vector is in the form of a plasmid, which is transferred into the target cells by one of a variety of methods: physical (e.g., microinjection, electroporation, scrape loading, microparticle bombardment or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

The instant invention also provides various methods for making and using the above-described genetically-modified cells. In particular, the invention provides a method for genetically modifying cell(s) of a mammalian recipient ex vivo and administering the genetically modified cells to the mammalian recipient. In one embodiment for ex vivo gene therapy, the cells are autologous cells, i.e., cells isolated from the mammalian recipient. As used herein, the term “isolated” means a cell or a plurality of cells that have been removed from their naturally-occurring in vivo location. Methods for removing cells from a patient, as well as methods for maintaining the isolated cells in culture are known to those of ordinary skill in the art.

The instant invention also provides methods for genetically modifying cells of a mammalian recipient in vivo. According to one embodiment, the method comprises introducing an expression vector for expressing a heterologous gene product into cells of the mammalian recipient in situ by, for example, injecting the vector into the recipient.

In one embodiment, the preparation of genetically modified cells contains an amount of cells sufficient to deliver a therapeutically effective dose of the therapeutic agent to the recipient in situ. The determination of a therapeutically effective dose of a specific therapeutic agent for a known condition is within the scope of one of ordinary skill in the art without the need for undue experimentation. Thus, in determining the effective dose, one of ordinary skill would consider the condition of the patient, the severity of the condition, as well as the results of clinical studies of the specific therapeutic agent being administered.

If the genetically modified cells are not already present in a pharmaceutically acceptable carrier they are placed in such a carrier prior to administration to the recipient. Such pharmaceutically acceptable carriers include, for example, isotonic saline and other buffers as appropriate to the patient and therapy.

The genetically modified cells are administered by, for example, intraperitoneal injecting or implanting the cells or a graft or capsule containing the cells in a target cell-compatible site of the recipient. As used herein, “target cell-compatible site” refers to a structure, cavity or fluid of the recipient into which the genetically modified cell(s), cell graft, or encapsulated cell expression system can be implanted, without triggering adverse physiological consequences

More than one recombinant gene can be introduced into each genetically modified cell on the same or different vectors, thereby allowing the expression of multiple therapeutic agents by a single cell.

The instant invention further embraces a cell graft. The graft comprises a plurality of the above-described genetically modified cells attached to a support that is suitable for implantation into a mammalian recipient. The support can be formed of a natural or synthetic material.

According to another aspect of the invention, an encapsulated cell expression system is provided. The encapsulated system includes a capsule suitable for implantation into a mammalian recipient and a plurality of the above-described genetically modified cells contained therein. The capsule can be formed of a synthetic or naturally-occurring material. The formulation of such capsules is known to one of ordinary skill in the art. In contrast to the cells that are directly implanted into the mammalian recipient (i.e., implanted in a manner such that the genetically modified cells are in direct physical contact with the cell-compatible site), the encapsulated cells remain isolated (i.e., not in direct physical contact with the site) following implantation. Thus, the encapsulated system is not limited to a capsule including genetically-modified non-immortalized cells, but may contain genetically modified immortalized cells.

Formulations of Therapeutic Compounds and Methods of Administration

Therapeutic compounds may be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

“Pharmaceutically acceptable” refers to molecular entities and compositions that are physiologically tolerated and do not typically produce an allergic or toxic reaction, such as gastric upset, dizziness and the like when administered to a subject or a patient. Exemplary subjects of the invention are vertebrates, mammals, and humans.

“Agent” herein refers to any chemical substance that causes a change. For example, agents include, but are not limited to, therapeutic genes, proteins, drugs, dyes, toxins, pharmaceutical compositions, labels, radioactive compounds, probes etc.

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The present invention also provides a method of delivering a gene encoding CHIP or functional subunit of CHIP to a target cell by producing viral particles, and then infecting the target cell with an effective amount of the infectious transgene vector particles. The target cell may be, for example, an airway epithelial cell, a central nervous system cell, or a hepatocyte cell.

The present invention also provides a method of producing in the form of infectious particles a transducing gene transfer vector containing a gene encoding CHIP or functional subunit of CHIP, by transfecting a packaging cell as described above with a packaging vector, and a transgene vector containing the remedial gene and a functional packaging signal, which by itself is incapable of causing a cell to produce transducing transgene vector particles, wherein the cell produces infectious transducing vector particles containing the transducing transgene vector in RNA form, a Gag protein, a Pol protein, pseudotyped with an envelope glycoprotein.

In yet another aspect, the invention features a method for treating a mammal diagnosed with a neurogenetic disorder. The method can include administering to the mammal a vector encoding CHIP or functional subunit of CHIP. The neurogenetic disorder can be a lysosomal storage disease, Huntington's disease, Parkinson's disease, amyotrophic lateral sclerosis, an ataxia, dentatorubral-pallidoluysian atrophy, prion disease, or Alzheimer's disease.

The invention will now be illustrated by the following non-limiting Examples.

### EXAMPLE 1

### CHIP Suppresses Polyglutamine Aggregation and Toxicity In Vitro and In Vivo

Materials and Methods

Plasmid Construction. The Q19, Q35, Q56, and Q80-GFP, GFPu, and CHIP plasmids were previously described (Connell et al., (2001) Nat Cell Biol 3:93-96; Meacham et al., (2001) Nat Cell Biol 3:100-105; Onodera et al., (1997) Biochem Biophys Res Commun 238:599-605; Bence et al., (2001) Science 292:1552-1555). To construct Q19-GFPu and Q80-GFPu the polyQ fragment and part of the EGFP coding sequence was cut from Q19 and Q80-GFP as a NheI to BsrgI fragment and ligated into the GFPu plasmid digested with NheI and BsrgI. Plasmids were verified by partial sequencing and immunoblotting with anti-GFP and 1 C2 antibodies (Trottier et al., (1995) Nature 378:403-406). GFP-Q82-Htt is an in-frame N-terminal fusion of GFP to truncated human huntingtin comprising human exons 1-3. The fusion protein was generated using recombinant PCR and the final 1.4 kb product was cloned into a pcDNA3.1.

Cell Culture and Transfections. All transfections were performed as previously described using Lipofectamine Plus™ (LifeTechnologies) (Pittman et al., (1993) J Neurosci 13:3669-3680; Chai et al., (1999) Hum Mol Genet 8:673-682). For experiments assessing aggregate suppression, 1 μg of plasmid encoding the polyQ proteins was co-transfected with 2 μg of CHIP or pcDNA3 as an empty vector control. For experiments using geldanamycin (Sigma St. Louis, Mo.) drug was dissolved in DMSO to a concentration of 360 μM and applied as described (Sittler et al., (2001) Hum Mol Genet 10: 1307-1315). Lactacystin (Calbiochem®, San Diego, Calif.) was added at a concentration of 10 μM 18 h after transfection and continued for 30 h.

Western Blotting and Immunofluorescence. Cos-7 cells were harvested 48-72 hours after transfection and subjected to western blot analysis as previously described (Chai et al., (1999) Hum Mol Genet 8:673-682). 1C2 antibody (1:1000 dilution) was used to detect the polyQ epitope in GFP-Q82-Htt. Q19 and Q71-GFPu were detected with anti-GFP (1:1000 dilution Medical and Biological Laboratories, Naka-ku Nagoya, Japan). CHIP was detected using either anti-CHIP polyclonal to full length recombinant CHIP (Oncogene Research Products, San Diego, Calif.) or an anti-CHIP peptide antibody raised against amino acids 218-232 of human CHIP (Abcam, Cambridge, Mass.) both at 1:1000 dilution. Hsp70 was detected using a polyclonal Hsp70 antibody (1:5,000 Stressgen, Victoria, British Columbia). Anti-alpha-tubulin mouse monoclonal antibody (1:10,000 Sigma, St. Louis, Mo.) was used as a loading control.

Cos-7, PC12 or primary neuronal cells were grown on coverslips and immunofluorescence for CHIP was carried out using an anti-CHIP peptide antibody raised against amino acids 218-232 of human CHIP (1:50 Abcam, Cambridge, Mass.). Fluorescence was detected using rhodamine conjugated secondary antibody (1:1000). Fluorescence was visualized with a Zeiss (Thornwood, N.Y.) Axioplan fluorescence microscope (magnification 630× and 1000×). Images were captured digitally with a Zeiss MRM AxioCam camera and assembled in Photoshop® 6.0 (Adobe® Systems, Mountain View, Calif.).

Pulse/Chase Experiments. Transfected Cos-7 cells were labeled with 225 μCi ³⁵S-Methionine for 30 min, washed 3 times and chased with media containing 45 mg/L unlabeled methionine for the indicated times. Cells were lysed under non-denaturing conditions in RIPA buffer with protease inhibitors (PI). GFP-Q82-Htt was immunoprecipitated with 2.5 μg anti-GFP antibody (Roche, clones 7.1 & 13.1) for 1 h at 4° C. and washed 4 times with RIPA+PI. Proteins were resolved in 4-15% gradient SDS-PAGE (Bio-Rad) and visualized with autoradiography.

Zebrafish Injections and Culture. Embryos were collected 10-20 minutes following natural matings. Plasmids were injected into the cytoplasm of single celled embryos using a PLI-90 picoinjector (Harvard Apparatus, Holliston, Mass.) and individually calibrated glass needles. Phenol red was added to plasmid solutions to monitor injections. For monitoring toxicity and inclusion formation of Q(n)-GFP proteins, 1 nl of a 250 ng/μl solution was injected. For double plasmid injections 1 nl of solution consisting of 200 ng/μl polyQ and 400 ng/μl CHIP was injected. Embryos were cultured at 28.5° C. in standard system water supplemented with 2% penicillin and streptomycin. Embryos that completely lysed in their chorions were scored as dead. This represented the overwhelming majority of embryos scored as dead. In cases where embryos were severely malformed but not lysed, the presence of visible beating of the heart tube was used to determine live/dead status. Less than 10% of embryos scored as dead fell into this second category. Statistical significance in mortality was determined via a two-tailed student's T-test with P<0.05 considered significant.

In Situ Lysis Assays. Cells expressing GFP-Q82-Htt were lysed 72 h after transfection by addition of 1.25% SDS and 1.25% triton-X-100 in PBS. Images were collected after 3 min of detergent treatment. Fluorescence was quantitated by collecting 3 non-overlapping images per well in each of two independent experiments. Pixel count was determined by highlighting all green pixels using the magic wand tool in Adobe® Photoshop® 6.0. Green pixels per image were determined using the histogram function. Average number of green pixels for cells transfected with GFP-Q82-Htt and empty vector was set at 100%.

Primary Neuronal Culture. Cultures were derived from cerebri of E16 wild-type B6C3F1/J mice (The Jackson Laboratory, Bar Harbor, Me.) as described previously (Meberg and Miller, Methods Cell Biol. 2003; 71:111-27) with minor modifications. Neurons were transfected using Lipofectamine Plus (Invitrogen®) 2-4 days after plating. Statistically significant differences in sick/dead cells were determined via a two-tailed student's T-test.

The vital dye exclusion assay was performed by adding 1.0% (w/v) trypan blue (Sigma, St. Louis, Mo.) dissolved in PBS to 1 ml culture media to achieve a final concentration of 0.01%. Identical microscopic fields were imaged before and 3 min after the addition of trypan blue.

Mouse Breeding. Mice were fed food and water ad libitum and maintained under 12 h light/dark lighting conditions. As HD mice became symptomatic, gruel was provided on the cage floor to supply adequate hydration and nutrition. N171-82Q HD transgenic mice were purchased from The Jackson Laboratory (Bar Harbor, Me.; B6/C3F1/J mixed background), while CHIP knockout mice were provided by Dr. Cam Patterson (University of North Carolina, Chapel Hill, N.C.; 129/C57B16 mixed background) (Dai et al., (2003) Embo J 22:5446-5458). HD transgenic male mice were bred to CHIP haploinsufficient (+/−) female mice to produce HD×CHIP^{+/-} offspring.

Tissue Preparation and Histochemistry. Mice were given an intraperitoneal injection of 200 μg/g ketamine and 20 μg/g xylazine. Transcardial perfusion of ice-cold 0.1 M phosphate buffered saline (PBS) pH 7.4 was followed by perfusion with an ice-cold fixative containing 4% paraformaldehyde in PBS (PFA/PBS) pH 7.4. Brains were removed and immersed in PFA/PBS overnight at 4° C., then placed in a 30% (w/v) sucrose PBS solution overnight at 4° C. for cryoprotection. Brains were embedded in OCT media and 30 μm-thick coronal sections were cut using a cryostat. Free floating sections were collected in 0.1 M PBS for staining.

Immunofluorescence on free-floating sections was done by incubating sections overnight in primary antibodies-mouse EM48 antibody (1:250) for Htt, and anti-ubiquitin (1:200 DAKO Carpinteria, Calif.). Fluorescence was detected using Cy3 or Cy5 conjugated secondary antibodies (1:200) incubated for 2 hours at room temperature. Blinded immunostaining and analysis of DARPP-32 was performed by peroxidase-based immunohistochemistry. Free-floating sections were incubated in 3% H₂O₂, followed by DARPP-32 primary antibody (1:1000; 24 h, RT; Chemicon International, Temecula, Calif.). After incubation in biotinylated goat anti-rabbit secondary antibody (1:200; 2 h, RT) sections were placed in an avidin-biotin HRP complex (ABC kit, 1 h, RT; Vector Lab, Burlingame, Calif.). Labeling was visualized using diaminobenzidine (DAB), each condition having an identical exposure time. High power brightfield was visualized with a Zeiss (Thornwood, N.Y.) Axioplan fluorescence microscope and images were captured digitally with a Zeiss MRM AxioCam camera. Whole slides were scanned electronically for digital imaging and images were assembled in Photoshop® 6.0 (Adobe Systems, Mountain View, Calif.).

Rotarod experiments. Rotarod testing was performed as described previously (Schilling et al., (1999) Hum Mol Genet 8:397-407) with minor modifications. Approximately four month-old (14-16 weeks) mice of each of the following genotypes were compared to age-matched wild-type mice: HD, CHIP^{+/-}, HD×CHIP^{+/-}. Mice were tested on an accelerating Rotarod device (Rota-Rod, Ugo Basile, Comerio VA, Italy). The speed was set to accelerate from 3 to 30 RPM over the course of five minutes. Mice were trained for one day prior to testing by running on the rotarod for two five-minute periods to familiarize them with the machine. Subsequent to training, three trials per day were performed over a three-day period. Latency times were recorded when mice either fell from the rod or passively rotated around the rod at least two times in succession. Each mouse had at least 10 minutes of recovery time between trials. The data for each genotype was averaged and plotted.

Results

CHIP Reduces Aggregation and Increases Solubility of Mutant PolyQ Protein.

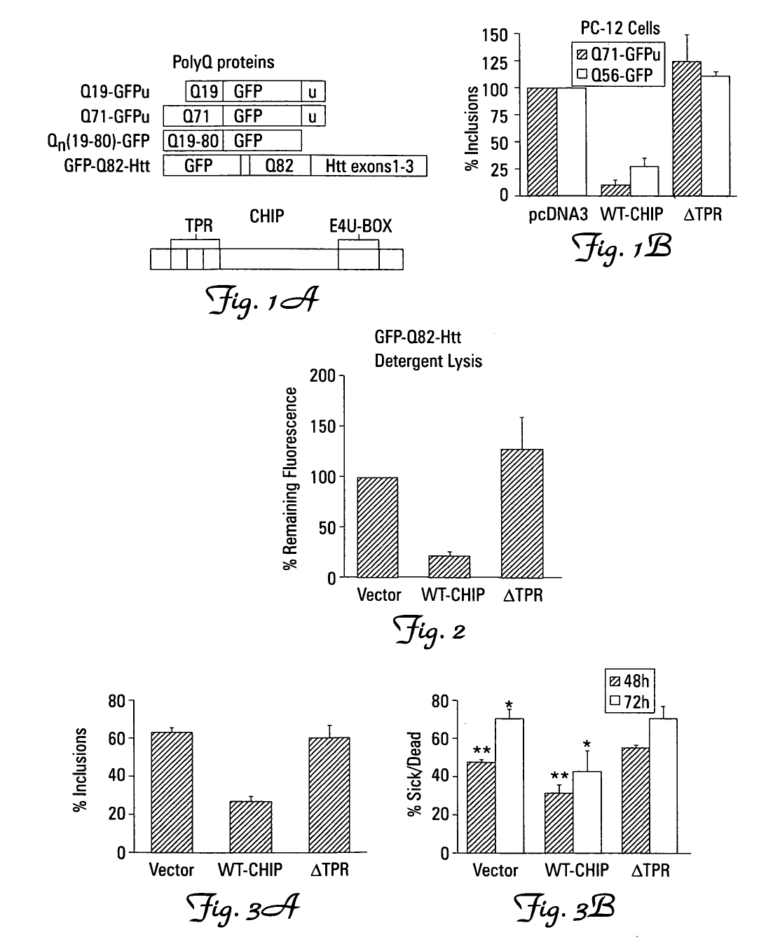

To test the hypothesis that CHIP modulates protein aggregation, mutant polyQ protein was co-expressed with wild type CHIP (WT-CHIP) or mutant CHIP deleted of either its N-terminal TPR domain (ΔTPR-CHIP) or C-terminal E4/U-box domain (ΔE4/U-box). Initially a widely used polyQ fragment was employed (Onodera et al., (1997) Biochem Biophys Res Commun 238:599-605) containing a normal or expanded repeat (Q19 or Q71). PolyQ fragments are extremely stable proteins when fused to conventional GFP (unpublished results), thus the polyQ domain was fused to degron-modified GFP (GFPu) in order to more accurately simulate normal protein processing while retaining the desirable qualities of bright fluorescence and rapid aggregation (FIG. 1A) (Onodera et al., (1997) Biochem Biophys Res Commun 238:599-605; Bence et al., (2001) Science 292:1552-1555).

Most Cos-7 cells expressing Q71-GFPu formed large juxtanuclear inclusions with little residual diffuse fluorescence, indicating that the polyQ protein had aggregated. In contrast, cells co-expressing WT-CHIP with Q71-GFPu had far fewer and smaller inclusions, with most GFP signal remaining diffuse. Consistent with these results, western blot analysis showed that co-expression of WT-CHIP caused more Q71-GFPu to migrate as soluble monomer in the separating gel with less of the polyQ protein remaining in the stacking gel as insoluble, aggregated material. Expression of a ΔTPR-CHIP mutant, which cannot interact with chaperones, did not reduce aggregates. In contrast, expression of the ΔE4/U-box mutant, which lacks E3 ligase activity, reduced inclusions and aggregates in a manner similar to WT-CHIP. The nonpathogenic fusion protein Q19-GFPu never formed aggregates and its gel migration and protein levels were unchanged by co-expression of any form of CHIP. These results indicate that the co-chaperone function of CHIP, mediated by its TPR domain, is required for suppression of polyQ aggregation. Its E3 ligase activity, however, appears to be dispensable.

Whether CHIP could inhibit inclusion formation in differentiated PC12 neural cells was also tested (FIG. 1B). PolyQ protein expression is less robust in PC12 cells than in Cos-7 cells and inclusion formation is accordingly less pronounced. CHIP activity was tested against GFP fusion proteins with or without a degron. Co-expression of WT-CHIP markedly suppressed the number of inclusion-containing cells, whether they expressed Q71-GFPu (7.4% of control levels) or the degron-less Q56-GFP (26.5% of control levels). Again, co-expression of ΔTPR-CHIP did not suppress aggregation of either polyQ protein (FIG. 1B). These results show that CHIP is capable of reducing polyQ protein aggregation in differentiated neural cells and that this effect does not depend on the presence of a degron.

Next, the ability of CHIP to suppress aggregation of expanded polyQ in the context of the HD protein huntingtin (Htt) was tested. A plasmid encoding the first three exons of Htt fused to GFP (GFP-Q82-Htt) was co-transfected together with WT-CHIP or ΔTPR CHIP plasmids. GFP-Q82-Htt formed large cytoplasmic inclusions. Co-expression of CHIP with GFP-Q82-Htt resulted in fewer and smaller inclusions with greater diffuse fluorescence in the rare, remaining inclusion-positive cells. Consistent with this result, WT-CHIP increased the amount of GFP-Q82-Htt migrating as soluble monomer on gels and decreased the amount of aggregated material retained in the stack. ΔTPR-CHIP again failed to suppress inclusion formation and aggregation (FIG. 2).

To quantify Htt aggregation, an in situ, detergent lysis technique was adopted. In this assay, fluorescence from soluble polyQ-GFP is quenched by the detergent but fluorescence from aggregated, insoluble protein is not (Kazantsev et al., (1999) Proc Natl Acad Sci USA 96:11404-11409). After detergent lysis, cells co-expressing WT-CHIP retained much less residual GFP fluorescence than cells co-transfected with ΔTPR-CHIP or empty vector (FIG. 2), confirming that CHIP reduced the formation of GFP-Q82-Htt aggregates.

From these cell-based studies, CHIP suppresses the formation of insoluble aggregates by mutant polyQ proteins. This property of CHIP requires an intact TPR domain, suggesting that interactions between CHIP and chaperones increase cellular capacity to appropriately handle misfolded polyQ proteins.

Aggregate Suppression by CHIP is Independent of Chaperone Induction or Enhanced Degradation.

Next, potential mechanisms by which CHIP reduces polyQ aggregation were examined. One possible explanation is that CHIP, through its interaction with HSF1, simply increases Hsp chaperone levels. Accordingly, the ability of CHIP to induce a stress response was tested and compared the results to those obtained with geldanamycin, a well-documented inducer of the stress response. Geldanamycin is a chemical inhibitor of Hsp90 that upregulates HSF1 activity and induces a stress response (Sittler et al., (2001) Hum Mol Genet 10:1307-1315). Cells with Q71-GFPu were co-transfected with either WT-CHIP or empty vector, then treated with or without geldanamycin. Geldanamycin itself reduced aggregation of Q71-GFPu, and induced a mild stress response exemplified by increased Hsp70 levels. Although CHIP also reduced aggregation in this assay, it did not induce Hsp70 under the same conditions. Moreover, there was no apparent synergy between CHIP and geldanamycin with respect to aggregate suppression or to Hsp70 induction. This suggests that CHIP does not suppress aggregation by inducing a stress response.

Whether CHIP acted by increasing the rate of turnover of mutant polyQ proteins was then tested. Cells were transfected with Q71-GFPu with or without WT-CHIP, then cells were treated with or without the proteasome inhibitor lactacystin. In the absence of co-expressed CHIP, polyQ aggregate formation was increased by lactacystin consistent with earlier reports. Interestingly, CHIP suppressed polyQ aggregation even in the presence of this proteasome inhibitor. To directly measure protein turnover, ³⁵S-methionine pulse-chase labeling was performed in cells expressing GFP-Q82-Htt with or without WT-CHIP. CHIP did not increase the rate of Htt degradation though it did enhance degradation of a well known substrate, CFTR (Meacham et al., (2001) Nat Cell Biol 3:100-105). Based on these experiments, enhanced degradation is not the primary route by which CHIP suppresses polyQ aggregate formation. Rather, this suppression likely involves enhanced Hsp70-dependent refolding or increased trapping of substrates in association with Hsp70. This is consistent with the previously reported effects of CHIP on folding of heat denatured luciferase (Kampinga et al., (2003) Mol Cell Biol 23:4948-4958).

CHIP Rescues Inclusion Formation and Toxicity in Primary Neurons.

Given the key role of polyQ oligomerization in triggering toxicity (Sanchez et al., (2003) Nature 421:373-379), it was hypothesized that CHIP's ability to suppress aggregation might translate into an ability to suppress neurotoxicity. This hypothesis was tested in primary neuronal cultures. CHIP is expressed in primary neurons and adult mouse brain, as confirmed by immunostaining and western blot analysis. In murine cortical neurons transfected with Q71-GFPu, large cytoplasmic and small neuritic inclusions with rare intranuclear inclusions were observed. MAP2 staining confirmed the neuronal identity of transfected cells. In cells transfected with Q71-GFPu and empty vector, inclusions were often associated with rounded cell bodies, retracted neurites and nuclear fragmentation. Upon co-expression of WT-CHIP, Q71-GFPu expressing cells were much more likely to maintain a typical neuronal appearance with less frequent inclusions and nuclear fragmentation. CHIP co-expression resulted in a 60% reduction in the number of neurons having inclusions (25% of neurons transfected with Q71-GFPu and CHIP versus 63% of neurons transfected with Q71-GFPu alone) (FIG. 3A). Neither endogenous nor overexpressed CHIP co-localized to inclusions (data not shown). ΔTPR-CHIP again failed to suppress inclusion formation, confirming the importance of the TPR domain for suppression of aggregation in neurons.

Next, whether suppression of aggregation by CHIP resulted in decreased neuronal cell death was determined. Cell morphology was examined under bright field and fluorescent illumination. Cells with flattened cell bodies and intact GFP-filled neurites were scored as living, whereas rounded cells with retracted neurites or cells displaying an apoptotic, blebbed morphology were scored as sick/dead. (Cells that could not be unambiguously scored were scored as living.) As shown in FIG. 3B, WT-CHIP conferred significant protection against polyQ toxicity (28% reduction in sick/dead cells by 72 hr; P<0.02 at both 48 and 72 hr), but ΔTPR-CHIP did not. To confirm the validity of the morphological criteria, transfected neurons were stained with trypan blue, a cell death detection dye that quenches GFP fluorescence in dead cells that are no longer able to exclude the dye. Consistent with the results in cell lines, overexpressed CHIP protected primary mammalian neurons from polyQ inclusion formation and toxicity in a manner dependent on the TPR domain of CHIP.

Zebrafish Model Recapitulates Features of PolyQ Disease.

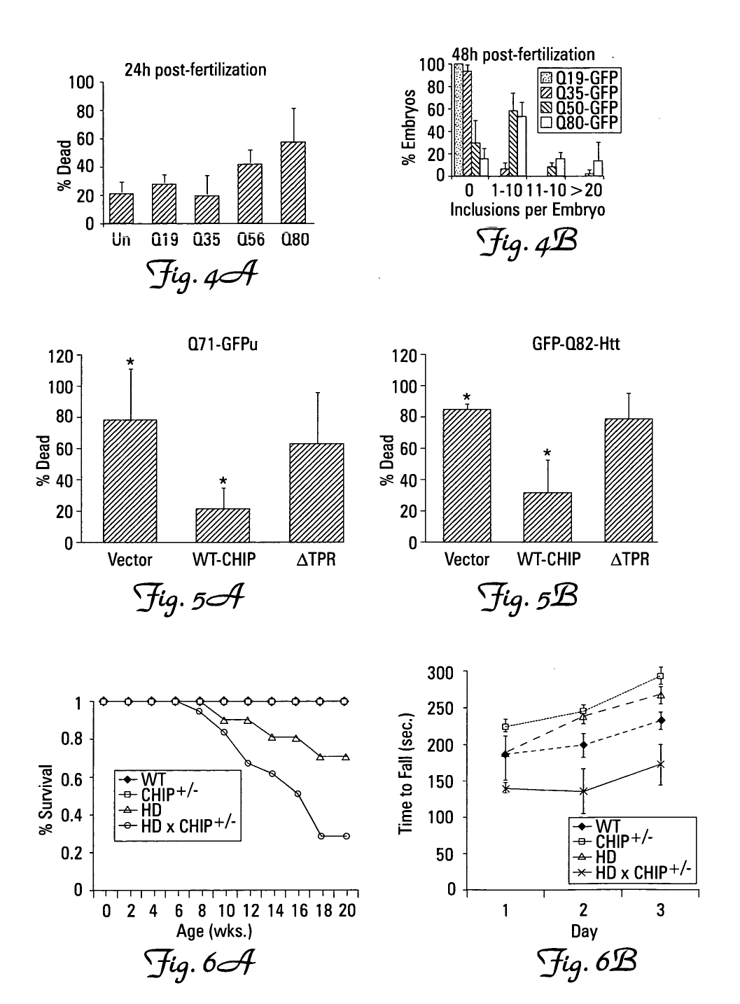

To develop a rapid, vertebrate-based, in vivo assay for testing potential disease modifiers like CHIP, the expression of polyQ proteins in zebrafish was explored. The effect of expressing various polyQ fusion proteins in zebrafish embryos was tested. Following fertilization, single-celled embryos were injected with plasmids encoding normal or expanded polyQ tracts fused to GFP (as shown in FIG. 1A). Developing embryos were monitored at 24 h and 48 h for toxicity and aggregation of the polyQ protein. At 24 h there were clear differences in the survival and morphology of embryos expressing pathogenic versus nonpathogenic polyQ tracts. Embryos expressing polyQ proteins with repeats below or near the disease threshold (Q19-GFP, Q35-GFP) were largely normal in appearance. There was slight developmental delay attributable to the injection procedure itself, but death was only slightly higher than in uninjected sibling embryos (FIG. 4A). In contrast, embryos expressing Q56-GFP or Q80-GFP were morphologically abnormal, displaying developmental delay and gross abnormalities in body plan and differentiation. There were also visible patches of brownish, opaque tissue containing rounded cells indicative of cell death. These cells stained with the cell death marker acridine orange. Death was elevated in Q56-GFP expressing embryos, and in the Q80-GFP injected group reached twice the level observed for Q19-GFP (FIG. 4A).

Inclusion formation by polyQ proteins in zebrafish embryos correlated with repeat length. At 48 h, surviving embryos were scored for the number of inclusion-containing cells per animal (FIG. 4B). The GFP-polyQ protein was diffusely distributed in embryos expressing polyQ repeats of nonpathogenic length. For example, Q19-GFP expressing embryos never contained inclusions, and only 7% of Q35-GFP expressing embryos had any inclusions. In contrast, Q56 and Q80-GFP expressing embryos had one or more inclusions in ˜70% and 85% of animals, respectively. Q80-GFP embryos also displayed many more inclusions per animal (FIG. 4B).

To test if these visible inclusions represented insoluble aggregates, an in situ lysis assay to assess the detergent solubility of inclusions was developed. This assay showed that pathogenic polyQ proteins form insoluble aggregates that can be monitored in developing fish.

Embryonic zebrafish recapitulate two important features of polyQ disease: repeat length-dependent toxicity and inclusion/aggregate formation. The repeat length for onset of pathology in zebrafish embryos parallels the threshold for polyQ length toxicity seen in human disease (Zoghbi et al., (2000) Annu Rev Neurosci 23:217-247). Moreover, the results with detergent lysis suggest that the sequential process of misfolding, oligomerization and aggregation occurs in zebrafish in a manner similar to that observed in disease tissue and other experimental systems (Sanchez et al., (2003) Nature 421:373-379).

CHIP Rescues PolyQ Toxicity in Zebrafish.

Zebrafish embryos were co-injected with plasmids expressing mutant polyQ proteins (Q71-GFPu and GFP-Q82-Htt) and either empty vector, WT-CHIP, or ΔTPR-CHIP. Embryos injected with mutant polyQ proteins and empty vector died at a high rate (FIGS. 5A and 5B). Most surviving embryos were severely disturbed in their overall body pattern, showed developmental delay and had prominent patches of dead or dying cells. Co-expression of WT-CHIP rescued polyQ-mediated death at 24 h, with the resultant embryos showing improved morphology although some developmental delay and occasional patches of dead and dying cells were still observed (FIGS. 5A and 5B). ΔTPR-CHIP co-expressing embryos died at the same frequency as polyQ protein-expressing embryos co-injected with control vector, and showed similar morphological disturbances among survivors (FIGS. 5A and 5B). Co-expression of CHIP decreased toxicity of both a generic polyQ-containing fragment and a pathogenic Htt fragment in zebrafish embryos. Thus, the ability of CHIP to reduce aggregation and toxicity of mutant polyQ proteins in cell lines and primary neurons correlates with suppression of toxicity in vivo.

CHIP Haploinsufficiency Accelerates PolyQ Disease in a Mouse Model of HD.

The above results suggested that endogenous CHIP plays an important role in buffering misfolded polyQ-induced toxicity in neurons. If CHIP assists in the neuronal handling of mutant polyQ protein, then reducing levels of CHIP would be predicted to exacerbate polyQ disease. To directly test this in vivo, the N171-Q82 HD transgenic mouse model (Schilling et al., (1999) Hum Mol Genet 8:397-407) was placed onto a CHIP haploinsufficient background (Dai et al., (2003) Embo J 22:5446-5458).

In N171-Q82 mice (HD mice), the prion promoter drives neuronal expression of the same Htt fragment employed in the cell culture and zebrafish studies (though minus the GFP tag). In this mouse model, ubiquitin-positive inclusions can be detected in several brain regions, most prominently in the cortex and cerebellum. These mice fail to gain weight and progressively display an HD-like motor phenotype characterized by tremor, gait disturbance, abnormal clasping and hypoactivity leading to premature death between 5-6 months of age. Progressive motor abnormalities in this model of HD most likely reflect polyQ-induced neuronal dysfunction rather than neuronal cell death since neuronal loss is not evident (Schilling et al., (1999) Hum Mol Genet 8:397-407).

HD mice were crossed to CHIP haploinsufficient (+/−) mice rather than to CHIP knockout (−/−) mice because the latter are not recovered at Mendelian ratios and are fragile, being highly susceptible to heat shock and other stressors (Dai et al., (2003) Embo J 22:5446-5458). In contrast, CHIP^{+/-} mice are indistinguishable from wild-type littermates in their gross appearance, weight, behavior and lifespan. In CHIP^{+/-} mice, a reduction in brain levels of CHIP protein was confirmed by western blot analysis.

The course of disease in HD transgenic mice with CHIP haploinsufficiency (HD×CHIP^{+/-}) differed significantly from HD transgenic mice with normal CHIP activity (HD), as well as from WT and CHIP^{+/-} mice. Mice of all four tested genotypes (HD; HD×CHIP^{+/-}; CHIP^{+/-}; and WT mice) appeared normal until 7-9 weeks of age. By 10-14 weeks, however, HD×CHIP^{+/-} mice displayed prominent kyphosis, poor grooming, tremor at rest and with activity, unsteady gait affecting the hindlimbs primarily, and marked hypoactivity. Upon tail suspension, HD×CHIP^{+/-} mice clasped both the front and hindlimbs and would not release this posture spontaneously. In contrast, age and sex-matched HD mice displayed only a slight degree of open field hypoactivity, mild action tremor and occasional, reversible clasping (mainly of the forelimbs) without other symptoms. Intriguingly, HD×CHIP^{+/-} males had the most severe phenotype, developing motor abnormalities 2-4 weeks earlier than females. After onset, the phenotype of HD×CHIP^{+/-} mice progressed rapidly to death over a 2-4 week period. By 20 weeks, only 27% of HD×CHIP^{+/-} remained alive, in contrast to 70% of HD mice and 100% of WT and CHIP^{+/-} mice (FIG. 6A).

To further characterize the motor phenotype of these mice, rotarod analysis was performed at 14-16 weeks of age. HD×CHIP^{+/-} mice were very fragile at this stage; several died soon after the first rotarod training session. The analyzed cohort thus represents only those animals well enough to complete testing. Nevertheless, even these less overtly affected HD×CHIP^{+/-} mice consistently performed worse than all other genotypes on the accelerating rotarod (FIG. 6B).

The distribution and intensity of Htt inclusions in HD mice were compared versus HD mice with CHIP haploinsufficiency. Immunostaining of brain sections with EM48 and anti-ubiquitin antibodies revealed an increase in Htt inclusions in HD×CHIP^{+/-} mice, most notably in the cerebellar granule cell layer. An increase in diffuse nuclear localization of Htt fragment was also noted in the hippocampus of HD×CHIP^{+/-} mice. This result is consistent with data obtained in transfected primary neurons, in which expression of expanded polyQ-GFP fragment in CHIP-deficient neurons resulted in increased numbers of inclusion-containing cells (data not shown). Htt inclusions were heavily ubiquitinated in both HD and HD×CHIP^{+/-} brains, indicating that ubiquitination activity is not globally impaired in neurons missing a single CHIP allele.

Analysis of the anatomical integrity of HD×CHIP^{+/-} brain sections stained with hematoxylin and eosin revealed no gross brain tissue or neuronal cell loss when compared to WT, CHIP^{+/-} or HD mouse brain sections. In contrast, immunohistochemical analysis revealed a reduction in the levels of the dopamine- and cAMP-responsive phosphoprotein of 32 kDa (DARPP-32) in the striatum of HD×CHIP^{+/-} mice compared to HD mice. A progressive loss in striatal DARPP-32 immunoreactivity has been previously linked to nigro-striatal dysfunction in mouse models of HD (Bibb et al., (2000) PNAS 97:6809-6814; van Dellen et al., (2000) Nature 404:721-722). In agreement with the gross tissue analysis, the decrease in striatal DARPP-32 staining in HD×CHIP^{+/-} mice appeared to result from reduced neuronal staining rather than overt cell loss. In summary, CHIP haploinsufficiency exacerbates and accelerates the neuronal dysfunction and behavioral phenotype displayed by N171-Q82 HD mice.

These results are also presented in Miller et al., Journal of Neuroscience, 25(40), 9152-9161 (2005), which is incorporated in its entirety herein.

### EXAMPLE 2

### C-Terminus of Hsp70 Interacting Protein (CHIP) Suppresses Toxicity of the Polyglutamine Disease Protein Ataxin-3 In Vivo

Polyglutamine (polyQ) diseases are inherited neurodegenerative disorders characterized by neuronal dysfunction and cell death. Expanded polyQ disease proteins are prone to misfold and aggregate, and promote global cellular protein folding defects. Cells possess systems for managing misfolded proteins, including chaperone-mediated refolding and ubiquitin-dependent proteasomal degradation. These pathways have been implicated in polyQ disease pathogenesis. The C-terminus of Hsp70 interacting protein (CHIP) is a co-chaperone and ubiquitin ligase that acts as a triage factor in cellular protein quality control, helping to determine whether misfolded proteins get refolded or degraded. In the polyQ disorder Huntington's disease, CHIP protects against toxicity. In the present study, the role of CHIP in polyQ diseases was explored more generally by investigating its activity in Spinocerebellar Ataxia Type 3 (SCA3).

The investigators bred mice expressing expanded human ataxin-3 cDNA (the full-length MJD1a splice variant with a 71-glutamine repeat) under the control of the prion promoter (Q71-B mice) to mice deficient in CHIP, resulting in hemizygous Q71-B mice with zero, one or two alleles of CHIP (Q^{+/-}C^{-/-}, Q^{+/-}C^{+/-} and Q^{+/-}C^{+/+}, respectively). Hemizygous Q71-B mice with normal CHIP levels had no behavioral phenotype and displayed minimal neuropathological changes over time, whereas Q71-B mice lacking CHIP developed severe gait abnormalities and early mortality. Moreover, Q71-B mice on a CHIP-haploinsufficient background developed age-dependent rotarod performance deficits. Brain lysate supernatants from Q71-B mice with decreased or absent CHIP showed a marked increase in SDS-resistant ataxin-3 complexes on denaturing gel electrophoresis, suggesting a correlation between phenotypic severity and the accumulation of high molecular weight ataxin-3 microaggregates in the brain. In transfected cells the investigators also showed that another E3/E4 ubiquitin ligase, E4B, mediated ataxin-3 degradation, whereas CHIP did not. Taken together the results are consistent with a disease model whereby CHIP increases the solubility of ataxin-3 and prevents its aggregation with only secondary, indirect effects on disease protein stability.

In summary, in Q71-B mice that are normally asymptomatic, CHIP haploinsufficiency caused a motor phenotype, while complete loss of CHIP caused a severe neurologic phenotype. A reduction in CHIP resulted in increased, soluble ataxin-3 microaggregates. Increase in ataxin-3 microaggregates correlated with increased phenotypic severity. Further, CHIP does not mediate ataxin-3 degradation; a different ubiquitin ligase, E4B, may perform this function.

In conclusion, CHIP plays an important role in “handling” expanded polyQ proteins, including ataxin-3, and CHIP assists in refolding expanded ataxin-3 and preventing oligomerization in vivo, rather than promoting degradation. The present evidence suggests that misfolded ataxin-3 oligomers, rather than monomers or inclusion bodies, are associated with neuronal dysfunction. Thus, CHIP has a pivotal role in determining how neurons handle expanded ataxin-3, and that CHIP generally protects against polyQ-induced toxicity.

Materials and Methods

Mouse strains. Q71-B^{+/-} mice were bred to CHIP^{+/-} mice to acquire Q71-B^{+/-}/CHIP^{+/-} mice. Progeny were bred to CHIP^{+/-} mice to generate Q71-B^{+/-} mice with two, one or no copies of CHIP.

Behavioral Analyses.

Accelerating rotarod. Animals performed 3 trials, with 10 minutes rest between trials. Trials ended when animals fell off the rod or began passively rotating.

Open field. Naïve animals were placed into a (25 cm×25 cm) box for 30 minutes. Movements were quantified and recorded by ViewPoint software.

Brain lysates. Mice were perfused transcardially with PBS plus protease inhibitors. Brain lysates were centrifuged into supernatant and pellet fractions. For Western blot, samples were diluted in SDS with DTT, then sonicated and centrifuged, and run on 4-15% Tris-HCl gradient gel.

Transfection, pulse-chase and immunofluorescence. Cos7 cells were transfected with the indicated plasmids using Lipofectamine™ PLUS (Invitrogen™) according to manufacturer's protocol. For pulse-chase, cells were pulsed for 15 minutes in 35S-containing medium and chased in complete medium for the chase times indicated. For immunofluroescence, cells were fixed in 4% paraformaldehyde 24 hours after transfection, and labeled with either CHIP (Abcam) or E4B (BD Biosciences) antibodies and DAPI.

Results

Q71-B/CHIP mouse line. By 4 months, Q71-B mice lacking CHIP developed abnormal posture and splaying of hindlimbs. In contrast, Q71-B that were haploinsufficient or wild-type for CHIP, as well as nontransgenic mice with reduced CHIP, appeared healthy. Although nontransgenic CHIP^{-/-} mice can develop kyphosis, they had normal motor behavior and lifespan.

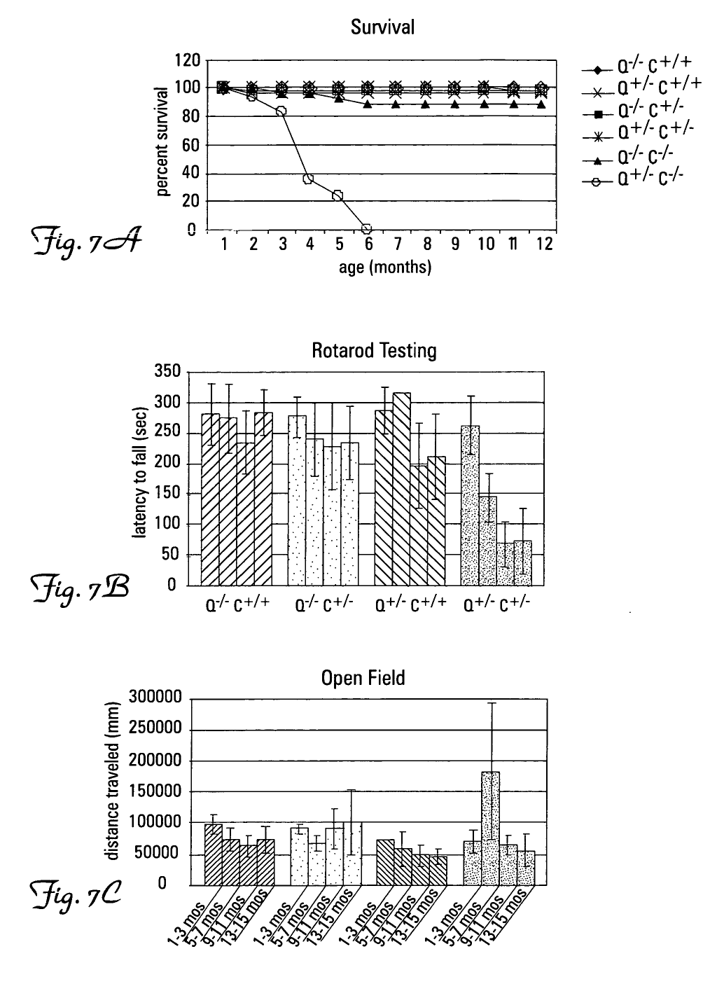

CHIP reduction increased mortality and caused motor deficits in Q71-B hemizygous mice. Q71-B mice lacking CHIP (Q^{+/-}C^{-/-}) exhibited early mortality and early-onset severe motor dysfunction precluding rotarod analysis (FIGS. 7A-7C). Q71-B mice with one copy of CHIP (Q^{+/-}C^{+/-}) developed mild motor dysfunction; however, there were no significant differences in open field behavior.

Loss of CHIP results in increased ataxin-3 microaggregates. Brain lysates from 4.5-month-old female mice demonstrated that depletion of CHIP (C^{+/-} or C^{-/-}) resulted in increased SDS-resistant, soluble ataxin-3. Removal of CHIP did not trigger aggregation of wild-type ataxin-3.

CHIP did not enhance ataxin-3 degradation, but another ubiquitin ligase, E4B, did. Pulse-chase experiments showed that overexpression of CHIP did not increase the degradation rate of FLAG-ataxin-3. Immunofluorescence on transfected Cos cells confirmed that CHIP overexpression did not reduce ataxin-3 levels. However, E4B overexpression did increase the degradation of FLAG-ataxin-3 (pulse-chase), and Cos cells overexpressing E4B had low ataxin-3 levels.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for decreasing the formation of an inclusion or aggregation of a target protein or for increasing the solubility of a target protein in a cell, comprising increasing the amount of C-terminal heat shock protein 70-interacting protein (CHIP) or a functional subunit of CHIP in the cell.

2. The method of claim 1, wherein an inclusion is decreased by 10% as compared to the cell prior to the increase in the amount of CHIP.

3. The method of claim 1, wherein aggregation of the protein is decreased by 10% as compared to the cell prior to the increase in the amount of CHIP.

4. The method of claim 1, wherein the solubility of the protein is increased by 10% as compared to the cell prior to the increase in the amount of CHIP.

5. The method of claim 1, comprising increasing the amount of CHIP or a functional subunit of CHIP by 10%.

6. The method of claim 1, comprising increasing the amount of a functional subunit of CHIP.

7. The method of claim 6, wherein the functional subunit of CHIP comprises at least one tetratrico peptide repeat (TPR) domain.

8. The method of claim 6, wherein the function subunit of CHIP comprises two TPR domains.

9. The method of claim 6, wherein the function subunit of CHIP comprises three TPR domains.

10. The method of claim 6, wherein the function subunit of CHIP does not comprise an E4/U-box domain.

11. The method of claim 1, wherein the target protein is a protein that comprises a polyglutamine repeat.

12. The method of claim 11, wherein the polyglutamine repeat has more than 46 glutamines.

13. The method of claim 1, wherein the cell is a mammalian cell.

14. The method of claim 1, wherein the cell is a neuronal cell.

15. The method of claim 1, wherein the cell is in vitro.

16. The method of claim 1, wherein the cell is in vivo.

17. The method of claim 16, wherein the cell is a neuron in a subject's brain.

18. The method of claim 1, wherein the amount of CHIP is increased by introducing a vector comprising a nucleic acid encoding CHIP into the cell.

19. The method of claim 18, wherein the vector is a viral vector or a plasmid.

20. The vector of claim 19, wherein the viral vector is an adenoviral vector, an adeno-associated virus vector, a recombinant lentivirus or retrovirus vector.

21. The method of claim 1, wherein the amount of CHIP is increased by introducing CHIP into the cell.

22. A method for treating a subject that has a neurodegenerative disease or for preventing a neurodegenerative disease in a subject, comprising administering to the subject a treatment effective to increase the amount of C-terminal heat shock protein 70-interacting protein (CHIP), or a functional subunit thereof, in cells of the subject.

23. The method of claim 22, wherein the subject is a mammal.

24. The method of claim 22, wherein the subject is not a human.

25. The method of claim 22, wherein the subject is a human.

26. The method of claim 22, wherein the neurodegenerative disease is a polyglutamine neurodegenerative disease.

27. The method of claim 22, wherein the neurodegenerative disease is Alzheimer's disease.

28. The method of claim 22, wherein the neurodegenerative disease is Huntington's disease.

29. The method of claim 22, wherein the increase in the CHIP or the functional subunit of CHIP is effective to decrease formation of inclusions in cells of the subject, to decrease aggregation of protein in cells of the subject, to decrease cell death of cells of the subject, or to increase the solubility of protein in cells of the subject.
